(19) United States
(12) Patent Application Publication
(10) Pub. No.: US 2026/0176586 A1
(43) Pub. Date: Jun. 25, 2026

(54) INJECTABLE THYMUS ORGANOIDS

(71) Applicant: FIBROBIOLOGICS, INC., Houston, TX (US)

(72) Inventors: Hamid KHOJA, Houston, TX (US); Simon GEBREMESKEL, Houston, TX (US); Pete O'HEERON, Houston, TX (US)

(21) Appl. No.: 19/544,833

(22) Filed: Feb. 19, 2026

Related U.S. Application Data
(63) Continuation of application No. PCT/US2025/033807, filed on Jun. 16, 2025.
(60) Provisional application No. 63/660,619, filed on Jun. 17, 2024.

(51) Int. Cl. C12N 5/078; A61K 9/00; A61K 35/26; A61K 35/33; C12N 5/071; C12N 5/077

(52) U.S. Cl. CPC C12N 5/065; A61K 9/0019; A61K 35/26; A61K 35/33; C12N 5/0656; C12N 5/0697; C12N 2510/00

(57) ABSTRACT

Provided here are thymus organoids, cell compositions, and pharmaceutical formulations thereof. Also provided are methods for using thymus organoids for clinical and non-clinical use.

### CROSS-REFERENCE TO RELATED APPLICATIONS

This application is a continuation of International Application No. PCT/US2025/033807, filed Jun. 16, 2025, which claims priority to U.S. Provisional Patent Application Ser. No. 63/660,619, filed 17 Jun. 2024, both of which are incorporated herein by reference in their entirety.

### BACKGROUND

### 1. Field

The present disclosure concerns at least the fields of cell biology, molecular biology, immunology, and medicine. The present disclosure provides cells and thymus organoid compositions, and methods of making and using the same, at least for the treatment of thymic disorders and immune related diseases.

### 2. Discussion of Related Art

The thymus is a lymphoid organ where precursors of T cells undergo differentiation, selection, and proliferation giving rise to diverse, functional, and self-tolerant T-cell populations. Age-related thymic involution is a natural process that leads to a gradual decrease in thymic size, function, and T cell production capabilities. In humans, age-related thymic involution begins after birth and the size of the thymus is thought to reach its peak during puberty, before gradually reducing in size. Thymic involution results in a progressive decline in the production of naïve T cells, reduction in TCR diversity, and impaired immune responses. In addition to this natural decline in thymic function, the proper functioning of the thymus can also be impaired by infection, exposure to chemotherapy or irradiation therapy, fluctuations in hormone levels, and many idiopathic causes. Decreased thymus function and output may result in susceptibility to cancer, autoimmune diseases, infections, and poor immunity from vaccination. Thymic involution has also been implicated in susceptibility to pathogen infections, tumors, and autoimmune diseases, and congenital disorders such as DiGeorge syndrome, congenital athymia, severe combined immune deficiency, FOXN1 deficiency, CHARGE Syndrome, and diabetic embryopathy. Therefore, there is a need in the field for off-the-shelf allogeneic strategies to rejuvenate the thymus and reinstate T cell production and function.

### SUMMARY OF THE DISCLOSURE

In an aspect the current disclosure encompasses a thymus organoid comprising (a) one or more fibroblasts and/or one or more fibroblast-derived products, and (b) one or more thymic epithelial cells, thymic epithelial progenitors, one or more thymocytes, or a combination thereof. In an aspect, the one or more fibroblasts may be engineered fibroblasts. The engineered fibroblasts may or may not encode at least one exogenous gene product. Non-limiting examples of at least one exogenous gene product include one or more Notch ligands, one or more nucleic acids, one or more cytokines, one or more chemokines, one or more transcription factors, one or more epigenetic factors, one or more growth factors, one or more hormones, any fragment or any derivative thereof, and/or a combination thereof. Examples of specific exogenous gene products include, but are not limited to DLL4, DLL1, DLL3, X-delta 2, JAG1, JAG2, FOXN1, IL-7, FLT-3L, SCF, CCL25, CXCL12, CXCL19, Lymphotoxin-alpha/beta, insulin-growth factor-1/2, fibroblast growth factor-7/10, FGF-21, HoxA1, Eya1, Pax9, Six1, Tbx1, Ripply3, E2F3, myc, miR-181a, Leptin receptor, ghrelin receptor, IL-22, a fragment thereof, a derivative thereof, or a combination thereof. In some aspects, the one or more fibroblasts can be from the skin and/or from the thymus of an individual, such as a human individual. In some aspects, the thymic epithelial cells may or may not be thymic epithelial progenitor cells. In some aspects, the fibroblasts, thymic epithelial cells, and/or thymocytes may or may not be differentiated from embryonic stem cells, induced pluripotent stem cells, hematopoietic stem cells, or progenitor cells.

In an aspect, the engineered fibroblast may or may not have a disrupted expression of one or more endogenous genes. In an aspect, the one or more thymic epithelial cells, one or more thymic epithelial progenitors, the one or more thymocytes, or a combination thereof are engineered cells. In an aspect, the engineered cell may have a disrupted expression of one or more endogenous genes. Non-limiting examples of endogenous genes that may be disrupted in any cells for use herein include androgen receptors selected from Pgr, Nrli2, Ar, Esr1, Esr2, Gper1, Nr3c1, Nr3c2, or a combination thereof. In an aspect, the endogenous gene comprises one or more immune check point genes and the engineered cell may be a thymocyte. In an aspect, the immune check point gene is selected from PD1, CTLA4, TIM3, TIGIT, CD96, BTLA, LAG3, or a combination thereof.

In an aspect, the cells included in the organoid may be engineered to disrupt at least the F3 gene encoding for tissue factor.

In an aspect, the ratio in the organoid of the one or more fibroblasts to the one or more thymic epithelial cells, one or more thymic epithelial progenitors, the one or more thymocytes, or a combination thereof is in the range of about 0.0001:1 to 10000:1, or 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1. In some aspects, the organoid has a size between 5 μm to 50,000 μm. In some aspects, the organoid has a size between 100 μm to 250 μm. In some aspects, the thymocytes may or may not comprise one or more T-cell progenitor cells and/or one or more mature T cell.

In an aspect, the organoid comprises one or more fibroblasts and/or one or more fibroblast-derived products, one or more thymic epithelial cells, one or more thymic epithelial progenitors, one or more T-cell progenitor cells or mature T cells, or any combination thereof, and the fibroblasts and/or one or more fibroblast, the one or more thymic epithelial cells, the one or more T-cell progenitor cells or mature T cells, or any combination thereof, are autologous, allogeneic, xenogeneic, or syngenetic with respect to an individual. In an aspect, the one or more fibroblasts, the one or more thymic epithelial cells, one or more thymic epithelial progenitors, the one or more T-cell progenitor cells or mature T-cells, or any combination thereof of the organoid are activated cells. In an aspect, the one or more fibroblasts, the one or more thymic epithelial cells, one or more thymic epithelial progenitors, the one or more T-cell progenitor cells, or any combination thereof are activated with one or more of nucleic acids, one or more cytokines, one or more chemokines, one or more transcription factors, one or more epigenetic factors, one or more growth factors, one or more hormones, or a combination thereof. In some aspects, the one or more fibroblasts, the one or more thymic epithelial cells, the one or more thymocytes, the one or more T-cell progenitor cells or a combination thereof may or may not be activated with CD3/28, concanavalin A, PMA/ionomycin, pokeweed mitogen, and/or PHA. In an aspect, the one or more fibroblasts are from thymic fibroblasts, embryonic stem cells, induced pluripotent stem cells, hematopoietic stem cells, and/or progenitor cells. In an aspect, the thymocyte comprises mature T-cells differentiated to target a single or multiple antigens. In some aspects, the antigen comprises gp100, MAGE-A, MART-1, HER2/neu, hTERT, CEA, p53, p21, CD19, CD20, GPC3, NYESO-1, MUC-1, CA-125, CA19-9, GD2, HPV (L1/E6,E7), HBV, SV40, CDk-4, beta-catenin, Mesothelin, PSMA, PTD52, Tyrosinase, PSA, PAP, EBV, KRAS, NRAS, cyclin B1, a mutant thereof, or a combination thereof.

In some aspects, disclosed is a pharmaceutical composition comprising a thymus organoid of the disclosure.

In an aspect, the current disclosure encompasses a method of generating a thymus organoid in vitro, comprising culturing a) the one or more fibroblasts and/or one or more fibroblast-derived products, with b) the one or more thymic epithelial cells, the one or more thymic epithelial progenitors, the one or more thymocyte, or any combination thereof in a culture medium, such as on a low adhesion surface. In an aspect, the culturing of the thymus organoid does not require an exogenous extracellular matrix. In an aspect, the culture medium is a serum-free medium. In an aspect, the culture medium comprises one or more nucleic acids, one or more cytokines, one or more chemokines, one or more transcription factors, one or more epigenetic factors, one or more growth factors, one or more hormones, or a combination thereof. In some aspects, the thymus organoid can generate thymic emigrant cells, immature T cells, mature T cells, antigen-specific T cells, chimeric antigen receptor (CAR)-T cells, CAR cells, regulatory T cells, cytotoxic T cells, invariant T cells, intraepithelial lymphocytes, gamma-delta T cells, or a combination thereof. In some aspects, a method of the disclosure further comprises the step of administering a composition of the disclosure (e.g., a thymus organoid) to an individual.

In an aspect, the current disclosure encompasses a method of regenerating thymus tissue and methods of treating a disease or disorder in a subject in need thereof, the method comprising administering to the subject an thymus organoid encompassed by the disclosure. In some aspects, the thymus organoid may be administered via an intravenous, intraarterial, subcutaneous, intraperitoneal, intramuscular, intrathymic and/or perithymic route. In an aspect, the administration of the thymus-organoid may or may not require a surgical procedure. In an aspect, the thymus organoid is administered via injection. In some aspects, the thymus organoid can be injected intravenously, intraarterially, subcutaneously, intraperitoneally, intramuscularly, intrathymically, intratumorally, peritumorally, and/or perithymically. Further aspects of the disclosure encompass a method of regenerating thymus tissue in a subject in need thereof, the method comprising administering to the subject one or more fibroblasts and/or one or more fibroblast-derived products; and (a) one or more thymic epithelial cells, and/or thymic epithelial progenitors, (b) one or more thymocytes, or (c) a combination thereof. In some aspects, the one or more fibroblasts, the one or more thymic epithelial cells, and the one or more thymocytes may be premixed and administered as a population of cells or a thymus organoid (or a combination thereof), or co-administered into a subject in need thereof. In some aspects, the subject may be diagnosed with or is suspected of having an immune deficiency, athymic diseases (DiGeorge syndrome, congenital athymia, severe combined immune deficiency, FOXN1 deficiency, CHARGE Syndrome, diabetic embryopathy, thymoma and thymic cancer associated diseases), acute or chronic thymic atrophy, thymic involution, cancer, autoimmune disease, infections, or any combination thereof. In some aspects, the subject may be administered at least one or more additional therapies. Non-limiting examples of the at least one additional therapy include chemotherapy, androgen deprivation therapy, immune activation therapy, cytokine therapy, checkpoint blockade therapy, and/or pain therapy.

### BRIEF DESCRIPTION OF THE DRAWINGS

The drawings form part of the present specification and are included to further demonstrate certain aspects of the present disclosure, which can be better understood by reference to the drawing in combination with the detailed description of specific aspects presented herein. The drawing figures do not limit the present disclosure to the specific aspects disclosed and described herein. The drawings are not necessarily to scale, emphasis instead being placed on clearly illustrating principles of certain aspects of the present concept.

FIG. 1 is a schematic showing a method of producing a three-dimensional thymus organoid by mixing dissociated thymus cells with fibroblasts.

FIG. 2 provides microscopic images of different sizes of three-dimensional thymus organoids.

FIG. 3 provides an immunofluorescence image showing spatial distribution of fibroblasts and Thymocytes in an organoid.

FIG. 4 provides FACS data to show viability of thymocytes and fibroblasts after 3 weeks in culture.

FIG. 5 shows that thymus organoid can support the progression of prethymocytes to double positive (CD4+ CD8+) cells within 3 weeks of culture. The FACS was gated on live lymphocytes.

FIG. 6 provides FACS data showing viability of fibroblasts and thymocytes after freezing the thymus organoids.

FIG. 7A is a schematic of the timeline of experiments following in vivo delivery of thymus organoids via subcutaneous, intraperitoneal, or intravenous routes into immunodeficient SCID mice.

FIG. 7B-7F provide representative flow cytometry plots on day 14 showing T cell frequencies in peripheral blood of FIG. 7B: wild-type, FIG. 7C: SCID mice, or SCID mice transplanted with thymus organoids, FIG. 7D: subcutaneously, FIG. 7E: intraperitoneally, or FIG. 7F: intravenously. FIG. 7G provides a summary of T cell frequencies on day 14 in the peripheral blood of wild-type, SCID, or SCID transplanted with thymus organoids subcutaneously, intraperitoneally, or intravenously.

FIG. 8A-8D provide a summary of T cell frequencies on day 25 in the peripheral blood of wild-type, SCID, or SCID transplanted with thymus organoids injected subcutaneously, intraperitoneally, or intravenously. FIGS. 8A and 8B are bar graphs showing the % T-cells and T-cell count in wild-type, SCID, or SCID transplanted mice respectively. FIGS. 8C and 8D are bar graphs showing the % T-cells and T-cell count in SCID, or SCID transplanted mice respectively, without the wild-type data, for clarity.

FIG. 9A provides a gating strategy for CD4+ and CD8+ T cells on day 25 in the peripheral blood of wild-type, SCID, or SCID transplanted with thymus organoids subcutaneously, intraperitoneally, or intravenously.

FIG. 9B-9C provides a summary of CD4+ and CD8+ T cell frequencies on day 25 in the peripheral blood of wild-type, SCID, or SCID transplanted with thymus organoids subcutaneously, intraperitoneally, or intravenously.

FIG. 9D-9E provides a summary of CD4+ and CD8+ T cell frequencies on day 25 in the peripheral blood of SCID, or SCID transplanted with thymus organoids subcutaneously, intraperitoneally, or intravenously.

FIG. 10 shows the gating hierarchy which is gated on CD45+ TCRb+ (T cells), then gated on CD4+ T cells, then gated on CD25+ FOXP3+ cells which are commonly referred to as T-regs or regulatory T cells.

FIG. 11 provides a schematic showing a method of producing a three-dimensional thymus nest from engineered fibroblast.

FIG. 12 provides a summary of NKT cells frequencies in the spleen of SCID mice transplanted with thymus organoids subcutaneously.

FIG. 13 provides a summary of gamma delta T cell frequencies in the spleen of SCID mice transplanted with thymus organoids subcutaneously.

FIG. 14 provides a summary of T cell frequencies from spleen of SCID mice transplanted with thymic organoids that were either freshly prepared or cryopreserved (“cryo”).

FIG. 15 shows a gene expression profile of thymus organoids. These are genes required to support T cell development and maturation.

FIG. 16 shows proliferation of T cell from SCID mice injected with thymus organoids and stimulated with CD3/28, Concanavalin A, or PHA.

FIG. 17 shows IFN-gamma production by T cells from SCID mice injected with thymus organoids and stimulated with CD3/28, Concanavalin A, or phytohemagglutinin (PHA).

FIG. 18 shows expression of various TCR—beta chains by T cells from SCID mice injected with thymus organoids.

FIG. 19 shows a subcutaneous thymus organoid 4 weeks after transplantation into a SCID mouse.

FIG. 20 shows CD3 staining (brown) in a subcutaneous thymus organoid 4 weeks after transplantation into a SCID mouse.

FIG. 21 shows viability of thymus organoids grown in media containing different concentrations of serum.

FIG. 22 shows tumor volume growth in SCID mice treated with or without thymus organoids comprising thymocytes engineered with a TCR targeting gp100.

FIG. 23 shows a three-dimensional human thymus organoid.

FIG. 24 shows organoids comprising fibroblasts and epithelial cells.

FIG. 25 shows cultures of fibroblasts, epithelial cells, and spheroids containing fibroblasts with epithelial cells.

### DETAILED DESCRIPTION

The following detailed description references the accompanying drawings that illustrate various aspects of the present concept. The drawings and description are intended to describe aspects and aspects of the disclosed concept in sufficient detail to enable those skilled in the art to practice the disclosed concept. Other components can be utilized, and changes can be made without departing from the scope of the present inventive concept. The following description is, therefore, not to be taken in a limiting sense. The scope of the present concept is defined only by the appended claims, along with the full scope of equivalents to which such claims are entitled.

The current disclosure is based in part on the unexpected discovery that fibroblasts mixed with other cells such as dissociated thymic stromal cells, including one or more of thymic epithelial cells, thymic epithelial progenitor cells, endothelial cells, mesenchymal cells, dendritic cells, and/or lymphocytes and including one or more T-cell progenitors, are capable of generating thymus organoids that at least can produce functional T cells in vitro and/or in vivo. In some aspects, the organoids may be generated without the need of one or more agents, such as added extracellular matrix, for example solubilized basement membrane like material (e.g., MATRIGEL®). In vivo studies showed that thymus organoids are capable of inducing and maintaining T-lineage differentiation and supporting survival, proliferation, and distribution of T-cells in immunodeficient mice, similar to a functional thymus. Interestingly, these organoids may be injected into a subject in need thereof, and do not necessarily require surgical transplantation. The organoids can also be easily stored and retain very high post storage viability. The organoids may comprise genetically and/or chemically modified cells. The ease of developing and using these thymus organoids make them a compelling and much needed clinical product. Additionally, the disclosed methods may be expanded to development of other organoids and tissue structures for clinical and non-clinical use.

Disclosed herein are compositions including cells and thymus organoids and methods of making and using the same for clinical and non-clinical purposes.

### I. Definitions

So that the present disclosure may be more readily understood, certain terms are first defined. Unless defined otherwise, all technical and scientific terms used herein have the same meaning as commonly understood by one of ordinary skill in the art to which aspects of the invention pertain. Many methods and materials similar, modified, or equivalent to those described herein can be used in the practice of the aspects of the present disclosure without undue experimentation, and particular materials and methods are described herein. In describing and claiming the aspects of the present disclosure, the following terminology will be used in accordance with the definitions set out below.

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1 %, etc. Whether or not modified by the term “about,” the claims include equivalents to the quantities.

As used herein, the term “activated immune cells” refers to immune cells treated with one or more stimuli capable of inducing one or more alterations in the cell: metabolic, immunological, epigenetic, growth factor secreting, surface marker expression, and production and excretion of microvesicles.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe, or using a surgical technique etc.

As used herein, “allogeneic” refers to tissues or cells from another body that in a natural setting are immunologically incompatible or capable of being immunologically incompatible, although from one or more individuals of the same species.

As used herein, “autologous” refers to tissues or cells that are derived or transferred from the same individual's body, for example tissues or cells transplanted from one part of an organism to another part of the same organism.

As used herein, “agent” refers to nucleic acids, cytokines, chemokines, transcription factors, epigenetics factors, growth factors, or hormones.

As used herein, “xenogeneic” refers to tissues or cells from a species different from the subject.

“Cell culture” is an artificial in vitro system containing viable cells, whether quiescent, senescent or (actively) dividing. In a cell culture, cells are grown and maintained at an appropriate temperature, typically a temperature of about 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “individual” or “subject”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (for example, children) and infants. It is not intended that the term “individual” connote a need for medical treatment, therefore, an individual may voluntarily or involuntarily be part of experimentation whether clinical or in support of basic science studies. The term “subject” or “individual” may be used interchangeably and refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “reduce,” “inhibit,” “diminish,” “suppress,” “decrease,” “prevent” and grammatical equivalents (including “lower,” “smaller,” etc. when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one aspect, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “unmodified” as used herein when referring to cells or a cell population may refer to cells that have only been cultured under standard conditions for the cell type, without exposure to any agent capable of changing the intrinsic characteristics (except those that may inherently change during a standard cell culture) of the cell type. An unmodified cell comprises only endogenous genetic material, i.e. an unmodified cell comprises no transgenes. In some aspects, it refers to cells that have not been manipulated in any way or that have only been cultured. In a specific aspect, “modified” cells are cells that have been stimulated to a point that they have differentiated into another cell type and may no longer have the morphology of the starting material. This can be applicable to MSCs and fibroblasts, as examples.

As used herein, the term “transplantation” refers to the process of taking living tissue or cells and implanting it in another part of the body or into another body.

The term “organoid” or “spheroid” may be used interchangeably herein and refer to a three-dimensional cell culture model that mimics the structure and function of an organ, and they may be derived at least from stem cells or organ progenitor cells in specific aspects. Organoids can recapitulate key features of the organ they represent, making them valuable tools for studying organ development, disease modeling, drug screening, and regenerative medicine.

Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment″ can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific aspects, treatment refers to the lessening in severity or extent of at least one symptom and may alternatively or in addition refer to a delay in the onset of at least one symptom.

The term “thymic disorder” encompasses any disease or disorder linked to the reduced or lack of one or more functions of the thymus in a subject. In an aspect, the disease or disorder may comprise immune deficiency, athymic diseases (DiGeorge syndrome, congenital athymia, severe combined immune deficiency, FOXN1 deficiency, CHARGE Syndrome, diabetic embryopathy), acute or chronic thymic atrophy, thymic involution, cancer, autoimmune disease, infections or any combination thereof.

As used herein the term “disrupted expression” encompasses both functional and structural disruption. Disrupted expression may refer to the permanent alteration or inactivation of a specific gene's function or expression, typically achieved through the introduction of insertions or deletions, or point mutations, at targeted genomic locus/loci within the gene or outside. In an aspect, the disrupted expression may result in complete loss of expression of a gene (knockout). In an aspect, the disrupted expression may result in reduction in the level of the specific gene product. Thus the gene may be expressed at less than 90%, less than 80%, less than 70%, less than 60%, less than 50%, less than 40%, less than 30%, less than 20%, less than 10%, less than 9%, less than 8%, less than 7%, less than 6%, less than 5%, less than 4%, less than 3%, less than 2%, less than 1% of its endogenous expression level in the cell or a control cell.

As used herein, the term “surgery” refers to a medical procedure involving the use of instruments to perform manual and operative techniques on a patient to investigate, treat a pathological condition such as disease or injury, or to help improve bodily function or appearance. The term specifically excludes procedures that involve only the injection of substances into the body with a needle or syringe, such as vaccinations or the administration of medications.

As used herein, the term “low adhesion surface” refers to a surface (e.g., a material, uncoated material, or coated material) to which a cell does not attach, substantially does not attach, or that substantially reduces the binding or adsorption of cell attachment proteins. A low adhesion surface can be hydrophilic and/or neutrally charged. Low adhesion surfaces can promote cell growth in a suspended state to facilitate organoid formation. A low adhesion surface can be a surface upon which less than 10%, less than 9%, less than 8%, less than 7%, less than 6%, less than 5%, less than 4%, less than 3%, less than 2%, less than 1%, less than 0.5%, less than 0.4%, less than 0.3%, less than 0.2%, or less than 0.1% cells attach to the surface.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous or exogenous genes or proteins that are not naturally expressed by the cells, either because the heterologous or exogenous proteins are recombinant or synthetic or because the cells do not naturally express the proteins.

### II. Compositions

### Thymus Cells and Organoids

In an aspect, the current disclosure encompasses a thymus organoid comprising one or more fibroblasts, and one or more thymic epithelial cells, one or more thymic epithelial progenitors, one or more thymocytes, or a combination thereof. The thymus organoid may further comprise additional cells which may or may not be associated with the thymus. Aspects of the disclosure relate to the formation of the three dimensional (3D) thymus organoid using fibroblasts alone or in combination with dissociated single cell from thymus tissue. These may include depletion of certain cell types from the thymus, and the enrichment of certain cell types from the thymus. In other aspects, there may be used fibroblasts in combination with epithelial cells, less differentiated cells such as hematopoietic stem or progenitor cells, pluripotent stem cells, and/or embryonic stem cells described herein and known in the art. In some aspects, the fibroblasts can be from the skin and/or from the thymus of an individual, such as a human individual. In some aspects, the fibroblasts derived products can be derived from fibroblasts from the skin and/or from the thymus of an individual, such as a human individual.

In some aspects, the one or more fibroblasts, one or more thymic epithelial cells, one or more thymic epithelial progenitors, and/or one or more thymocytes can be differentiated from one or more source cells (e.g., one or more progenitor cells and/or one or more stem cells). Differentiation of source cells can involve transforming pluripotent stem cells, such as embryonic stem cells, and/or progenitor cells into other specific cells, including fibroblasts, thymic epithelial cell, thymic epithelial progenitors, and/or thymocytes. In some aspects, a source cell includes embryonic stem cells, induced pluripotent stem cells, hematopoietic stem cells, thymic epithelial progenitor cells, and/or progenitor cells. This process can be orchestrated by exposing the source cells to various cytokines, chemokines, growth factors, transcription factors, and/or other stimulus that guide their development into desired cell types. The differentiated cells can be used to form thymus organoids. Differentiation protocols are known in the art. For example, IPSC can be differentiated into thymic epithelial cells as described in Ramos et al, Generation of functional human thymic cells from induced pluripotent stem cells. J Allergy Clin Immunol. 2022 February; 149(2): 767-781.e6. PMCID: PMC8815270 (incorporated herein by reference in its entirety). In some aspects, embryonic stem cells can be differentiated into thymic epithelial progenitors as described in Lai et al, Generation of thymic epithelial cell progenitors by mouse embryonic stem cells. Stem Cells. 2009 December; 27(12): 3012-20. doi: 10.1002/stem.238. PMID: 19824081 (incorporated herein by reference in its entirety). In some aspects, FOXN1 can be expressed in embryonic fibroblasts to induce differentiation into thymic epithelial cells as described in Oh et al, Thymic rejuvenation via FOXN1-reprogrammed embryonic fibroblasts (FREFs) to counteract age-related inflammation. JCI Insight. 2020 Sep. 17; 5(18):e140313. doi: 10.1172/jci.insight.140313. PMID: 32790650; PMCID: PMC7526556 (incorporated herein by reference in its entirety).

As used herein, the term fibroblast is applied broadly to include fibroblast cells and fibroblast-like cells from any tissue or organ. In some aspects, a fibroblasts is not a fibroblast-like cell. In general fibroblasts are connective tissue cells that synthesize extracellular matrix and collagen, thereby providing a structural framework. Therefore, the fibroblasts for use in the current disclosure can encompass any cell, or derivatives thereof, capable of providing an extracellular structural framework. The fibroblast may be isolated from a subject, or may be a cultured fibroblast. Fibroblasts may be isolated from an autologous, allogeneic, xenogeneic, syngenetic, or a combination thereof, with respect to the subject. In some aspects, fibroblasts are not mesenchymal stem cells. A fibroblast cell may or may not be from the skin, thymus, heart, blood vessels, bone marrow, skeletal muscle, liver, pancreas, brain, foreskin, omentum, adipose tissue, placenta, and/or umbilical cord, which can be obtained by biopsy (where appropriate) or upon autopsy. In some aspects, fibroblasts may or may not be from a fetal, neonatal, adult origin, or a combination thereof. In some aspects, fibroblasts may or may not be from mammals such as human, primate, porcine, bovine, murine, canine, and/or feline.

The term “fibroblast-derived product” as used herein refers to fibroblast cell fragments, conditioned media, biologic components, growth factors, cytokines, chemokines, lysates and/or extracellular vesicles secreted or isolated from fibroblasts. Fibroblast-derived extracellular vesicles can include exosomes, microvesicles, and/or apoptotic bodies. Fibroblast-derived fragment can include membranes. The fibroblasts derived products may have one or more characteristics of the fibroblasts from which they are derived, including, but not limited to, surface markers, proteins, and/or RNA.

In an aspect, the fibroblast may be differentiated in vitro from an autologous, allogeneic, xenogeneic, or syngenetic cell, for example a stem cell. In an aspect, the stem cell may be a pluripotent stem cell or a progenitor stem cell. In an aspect, the stem cells may be embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACC), umbilical cord stem cells, amniotic fluid stem cells, amnion derived adherent cells (AMDACs), osteogenic placental adherent cells (OPACs), limbal stem cells, dental pulp stem cells, endothelial progenitor cells, exfoliated teeth derived stem cells, hair follicle stem cells, dermal stem cells, parthenogenically derived stem cells, reprogrammed stem cells, amnion derived adherent cells, side population stem cells, or a combination thereof. In an aspect, the stem cells may be engineered to encode at least one exogenous gene product or have disrupted expression of an endogenous gene or both. The exogenous gene product may be notch ligands, nucleic acids, cytokines, chemokines, transcription factors, epigenetic factors, growth factors, hormones, and any combination thereof. Non-limiting examples of one or more encoded exogenous gene products include DLL4, DLL1, DLL3, X-delta 2, JAG1, JAG2, FOXN1, IL-7, FLT-3L, SCF, CCL25, CXCL12, CXCL19, Lymphotoxin-alpha/beta, insulin-growth factor-1/2, fibroblast growth factor-7/10, HoxA1, Eya1, Pax9, Six1, Tbx1, Ripply3, E2F3, myc, miR-181a, Leptin receptor, ghrelin receptor, IL-22, or a fragment thereof, or a derivative thereof, or a combination thereof. The stem cell may have disrupted expression of one or more endogenous genes, non-limiting examples of which include Pgr, Nrli2, Ar, Esr1, Esr2, Gper1, Nr3c1, Nr3c2 androgen receptor. As used herein the term disrupted expression encompasses both functional and structural disruption. Disrupted expression may refer to the permanent alteration or inactivation of a specific gene's function or expression, typically achieved through the introduction of insertions or deletions, or point mutations, at targeted genomic locus within the gene or outside. In an aspect, the disrupted expression may result in complete loss of expression of a gene (knockout). In an aspect, the disrupted expression may result in reduction in the level of the specific gene product. Thus, the gene may be expressed at less than 90%, less than 80%, less than 70%, less than 60%, less than 50%, less than 40%, less than 30%, less than 20%, less than 10%, less than 9%, less than 8%, less than 7%, less than 6%, less than 5%, less than 4%, less than 3%, less than 2%, less than 1% of its endogenous expression level in the cell. In an aspect, the disrupted expression may be a functional disruption, in that the gene product does not perform one or more of its endogenous functions or exhibits reduced functionality. For example, for an enzyme, this could manifest as an alteration in enzyme kinetics as measured by methods known in the art.

In an aspect, the fibroblasts may be isolated from an autologous, allogeneic, xenogeneic, syngenetic, or a combination thereof, with respect to the subject. The fibroblast may be isolated from any organ of the body. Fibroblasts may be from various tissues or organs, including, but not limited to skin, thymus, heart, blood vessels, bone marrow, skeletal muscle, liver, pancreas, brain, foreskin, which can be obtained by biopsy (where appropriate) or upon autopsy. In some aspects, a fibroblast is not from skin, thymus, heart, blood vessels, bone marrow, skeletal muscle, liver, pancreas, brain, or foreskin. In some aspects, the cells comprise fibroblasts, which can be from a fetal, neonatal, adult origin, or a combination thereof. In some aspects, a fibroblast is not from a fetal, neonatal, or adult origin. In an aspect, the one or more fibroblast is isolated from the thymic tissue. In an aspect the thymic fibroblasts may include capsular fibroblasts (Pdpn^{+ DPP}4⁺) and/or medullary fibroblasts (Pdpn^{+ DPP}4⁻). The one or more fibroblasts may be a modified fibroblast, or an unmodified fibroblast. In an aspect, the one or more fibroblasts may be an engineered fibroblast. In an aspect, the fibroblast may be engineered to encode at least one exogenous gene product or have disrupted expression of an endogenous gene or both. The exogenous gene product may be notch ligands, nucleic acids, cytokines, chemokines, transcription factors, epigenetic factors, growth factors, hormones, and any combination thereof. Non-limiting examples of one or more encoded exogenous gene products include DLL4, DLL1, DLL3, X-delta 2, JAG1, JAG2, FOXN1, IL-7, FLT-3L, SCF, CCL25, CXCL12, CXCL19, Lymphotoxin-alpha/beta, insulin-growth factor-1/2, fibroblast growth factor-7/10, HoxA1, Eya1, Pax9, Six1, Tbx1, Ripply3, E2F3, myc, miR-181a, Leptin receptor, ghrelin receptor, IL-22, or a fragment thereof, or a derivative thereof. The fibroblast may have disrupted expression of one or more endogenous genes, non-limiting examples of which include Pgr, Nrli2, Ar, Esr1, Esr2, Gper1, Nr3c1, Nr3c2 androgen receptor. In an aspect, the one or more fibroblasts may comprise an activated fibroblast. The fibroblast cells may be activated, such as having activated or engineered surface markers, nucleic acid modification, and/or expression or excretion of one or more chemokines, cytokines, exosomes, and/or growth factors. Methods of activating fibroblasts are well known in the art and may encompass contacting the one or more fibroblasts with one or more of nucleic acids, cytokines, chemokines, transcription factors, epigenetic factors, growth factors, hormones, or any combination thereof.

In an aspect, the disclosed organoid may further comprise one or more cell types in addition to the fibroblasts and (a) epithelial cells and/or epithelial progenitor cells, (b) thymocytes, or (c) combination thereof, for example cells typically found in the thymus. Non-limiting examples include thymic epithelial cells (TEC), endothelial cells, mesenchymal cells, dendritic cells, and/or thymocytes including one or more T-cell progenitors. Examples of thymic epithelial cells include squamous TEC, stellate TECs, medullary TECs, Hassall corpuscles, myoid cells, neuroendocrine cells. Examples of thymocytes include early thymic progenitors (ETPs), double-negative (DN) T cells, double-positive (DP) T cells, single-positive (SP) T cells, regulatory T cells (Tregs), invariant Natural killer T cells (Type I, II, and NKT-like) and intraepithelial lymphocyte precursor type A (IELpA) cells. In an aspect, the thymocytes are T-cells engineered or activated to target a single or multiple antigens. Methods of engineering or activating T-cells to target specific antigens are well known in the art.

In an aspect, the additional cells may include vascular smooth muscle cells (VSMCs), endothelial cells, lymphatic endothelial cells, dendritic cells, activated dendritic cells (aDCs), plasmacytoid dendritic cells (pDCs), monocytes, macrophages and megakaryocytes. In an aspect, the one or more additional cell is a TEC. In an aspect, the one or more additional cell is a thymocyte. In an aspect, the one of more additional cells may be activated, such as adding activating antigens, mitogens, having activated or engineered surface markers, nucleic acid modification, and/or expression or excretion of one or more chemokines, cytokines, exosomes, and/or growth factors. Methods of activating fibroblasts and/or cells are well known in the art and may encompass contacting the one or more fibroblasts with one or more of nucleic acids, cytokines, chemokines, transcription factors, epigenetic factors, growth factors, hormones, or any combination thereof. In an aspect, the thymus organoid may express one or more exogenous human major histocompatibility complex, exogenous co-stimulatory molecules, exogenous biological molecules, or genes that alter T cell differentiation, function, or activation.

In an aspect, the ratio of the fibroblast to the one or more additional cells may range from about 0.0001:1 to 10000:1. Thus, in an aspect, the fibroblast to other cells (e.g., TEC and/or thymocytes) ratio may vary from about 0.0001:1, 0.001:1, 0.01:1, 0.1:1, 1:1, 10:1, 100:1, 1000:1, 10000:1, 0.0002:1, 0.002:1, 0.02:1, 0.2:1, 2:1, 20:1, 200:1, 2000:1, 20000:1, 0.0005:1, 0.005:1, or any value within the range. In an aspect, the fibroblast to thymocyte ratio may vary from about 0.0001:1, 0.001:1, 0.01:1, 0.1:1, 1:1, 10:1, 100:1, 1000:1, 10000:1, 0.0002:1, 0.002:1, 0.02:1, 0.2:1, 2:1, 20:1, 200:1, 2000:1, 20000:1, 0.0005:1, 0.005:1, or any value within the range. In an aspect, the ratio of the fibroblasts to the one or more additional cells can vary from about 0.0001:1, 0.001:1, 0.01:1, 0.1:1, 1:1, 10:1, 100:1, 1000:1, 10000:1, 0.0002:1, 0.002:1, 0.02:1, 0.2:1, 2:1, 20:1, 200:1, 2000:1, 20000:1, 0.0005:1, 0.005:1, or any value within the range. In an aspect, the fibroblast to other cells (e.g., TEC and/or thymocyte) ratio can be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100 or any ratio therebetween. In an aspect, the fibroblast to thymocyte ratio can be 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1, or any ratio therebetween.

The disclosed organoid may be about 5 μm to 50,000 μm in size. In an aspect, the disclosed organoid may range in size from about 5 82 m-50 μm, about 50 μm-60 μm, about 60 μm-70 μm, about 70 μm-80 μm, about 80 μm-90 μm, about 90 μm-100 μm, about 100 μm-200 μm, about 100 μm-300 μm, about 100 μm-500 μm, about 100 μm-750 μm, about 100 μm-1,000 μm, about 100 μm-1,500 μm, about 1,500 μm-2,000 μm, about 2,000 μm-3,000 μm, about 3,000 μm-4,000 μm, about 4,000 μm-5,000 μm, about 5,000 μm-6,000 μm, about 6,000 μm-7,000 μm, about 7,000 μm-8,000 μm, about 8,000 μm-9,000 μm, about 9,000 μm-10,000 μm, about 10,000μm-20,000 μm, about 20,000 μm-30,000 μm, about 30,000 μm-40,000 μm, about 40,000 μm-50,000 μm in size.

In an aspect, the disclosed organoid may be for clinical or experimental use in a subject. In an aspect, the disclosed organoid may be developed for clinical use in a subject in need thereof. In an aspect, the one or more cells used to develop the organoid, for example, the one or more fibroblasts, the one or more TEC, the one or more thymocytes, or any combination thereof may be autogenic, allogeneic, xenogeneic, or syngenetic to the subject.

In specific aspects, the cells or organoids of the disclosure can be specifically formulated for administration into a subject in need thereof. They may or may not be formulated as a cell suspension. The cells/organoids may be formulated specifically for intravenous administration. comprising one or more pharmaceutically acceptable carriers, excipients, or vehicles suitable for delivering organoids to a subject. Such formulations may further comprise additives such as stabilizers, preservatives, or penetration enhancers to enhance the stability, bioavailability, or targeted delivery of the organoids. In an aspect, the disclosed compositions may be formulated to maintain cellular osmolarity, pH, and metabolic activity during storage. Salts, sugars like glucose, and buffering agents such as HEPES ensure the physiological integrity of organoids, minimizing ischemic damage and preserving their viability until transplantation or administration.

For injections of organoids and tissue mass, excipients are tailored to ensure optimal delivery and performance. Physiological saline solutions are frequently utilized as a vehicle for injections, maintaining tissue hydration and osmolarity while minimizing cellular stress. Buffered solutions, such as phosphate-buffered saline (PBS), Plasma-lyte A, Ringers solution or HEPES-buffered saline, serve to stabilize pH levels and osmotic balance, crucial for maintaining tissue integrity during the injection process.

To enhance stability and efficacy, stabilizers may be incorporated into injection formulations. Compounds like albumin or gelatin aid in preventing tissue aggregation or degradation, safeguarding the structural and functional integrity of injected tissues. The cell/organoid formulation may comprise albumin, including human albumin, with a specific formulation comprising 2.5% human albumin. Furthermore, penetration enhancers, such as surfactants or liposomes, may be included to facilitate tissue penetration and uptake of therapeutic agents, ensuring targeted delivery and efficacy.

To further prevent reduce potential innate or adaptive immune response, and prevent coagulation, the organoid maybe either pre-treated with heparin or administered along with heparin in order to block tissue factor receptor on cell surfaces of the organoid.

Overall, the selection and formulation of excipients for organoid and tissue storage and injection are guided by principles of biocompatibility, tissue compatibility, and regulatory requirements. Careful consideration of these factors, alongside thorough validation and testing, is essential to developing safe and effective formulations for preserving and delivering organs and tissues in clinical and research settings. The cells/organoids may be formulated specifically for intravenous administration; for example, they may be formulated for intravenous administration over less than one hour. The disclosed formulations find application in various therapeutic, diagnostic, or research contexts, including but not limited to tissue repair, organ regeneration, disease treatment, and imaging.

Additionally, methods of preparing, administering, and using said formulations, as well as kits comprising the same, are encompassed within the scope of the present disclosure. In specific cases they are formulated in a single dose form or multiple dose form. They may be formulated for systemic or local administration. In some cases, the cells are formulated for storage prior to use, and the cell formulation may comprise one or more cryopreservation agents, such as DMSO (for example, in 5% DMSO), methylcellulose, ethylene glycol, or any combination thereof. In particular aspects the cells are in a formulated cell suspension that is stable at room temperature for 1, 2, 3, or 4 hours or more from time of thawing.

### II. Methods

### Methods of Cell and Organoid Culture

In an aspect, the current disclosure also encompasses a method of generating thymus organoid(s) encompassed by the disclosure. The method may comprise culturing the (a) one or more fibroblasts, and (b) the one or more thymic epithelial cells (TEC), the one or more thymic epithelial progenitors (TEPs), the one or more thymocytes, or any combination thereof, on a low adhesion surface. As disclosed herein, any of the cells encompassed herein may be obtained from an endogenous source or cultured, and/or differentiated in vitro prior to mixing the cells with the disclosed fibroblasts. In an aspect, the method may comprise culturing the one or more fibroblasts as provided herein, with dissociated thymus from an autogenic, allogeneic, xenogeneic, or syngenetic source. Cells obtained from the dissociated thymus may be selectively used in the culture, or may be used as a thymic extract. Where the cells are selectively used, one or more isolated cells may be further proliferated, differentiated, or both prior to mixing the fibroblasts. In an aspect, the one or more additional cells may not be of thymic origin. As such the cells may be isolated or derived from cells isolated from various tissues or organs, including, but not limited to skin, heart, blood vessels, bone marrow, skeletal muscle, liver, pancreas, brain, foreskin, which can be obtained by biopsy (where appropriate) or upon autopsy. Derivative cells may also originate from cell-lines.

### Cell Culture Methods

As provided above, certain aspects of the method concern culturing the disclosed cells for incorporation into compositions and/or use in methods described herein. In some aspects, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often from animal blood, such as calf serum.

In some aspects, cells may be cultured for at least between about 10 days and about 40 days, for at least between about 15 days and about 35 days, for at least between about 15 days and 21 days, such as for at least about 15, 16, 17, 18, 19 or 21 days. In some aspects, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some aspects, a culture medium formulation may be explants medium (CEM) which is composed of IMDM supplemented with 10% fetal bovine serum (FBS), 100 U/ml penicillin G, 100 pg/ml streptomycin and 2 mmol/L L-glutamine. Other aspects may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (aMEM), and Roswell Park Memorial Institute Media 1640 (RPMI Media 1640) and the like. Typically, up to 20% fetal bovine serum (FBS) or 1-20% horse serum is added to the above medium in order to support the growth of cells. However, use of serum-free media, or media comprising cell extracts with no added serum is also envisaged. A defined medium can also be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure. For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular aspects, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### Organoid Generation

In an aspect, the current disclosure encompasses a method of generating a thymus organoid in vitro, the method comprising culturing one or more fibroblasts; and one or more additional cells, for example thymic epithelial cells (TEC), thymic epithelial progenitors (TEPs), endothelial cells, mesenchymal cells, dendritic cells, thymocytes, or any combination thereof, in a culture medium, on a low adhesion surface. In an aspect, the fibroblasts may be combined with enzymatically or non-enzymatically, or both, dissociated thymus and can be cultured together in vitro to form a thymic organoid. One of more cell types from the dissociated thymus may be depleted, and other cell types may be added. Additionally, the disclosure also encompasses culturing of fibroblasts with isolated endogenous or derivatized cells, including thymocytes, epithelial cells, endothelial cells, mesenchymal cells, dendritic cells, thymocytes, or any combination thereof.

In an aspect, the organoids may be developed in a coated dish, wherein the coating makes the dish low adhesion. Non-limiting examples of low adhesion cell culture dish coatings include polyethylene glycol (PEG), polyvinyl alcohol (PVA), Pluronic F-127, and hydrogels like agarose or alginate. Any suitable apparatus/dish may be used for developing the disclosed organoids. Non-limiting examples include petri dishes, multi-well plates, cell culture flasks, roller bottles, and bioreactors. Non-limiting examples of low adhesion dish include ELPLASIA® plates, ELPLASIA® flasks, CORNING® Ultra-Low Attachment Plates, GREINER CELLSTAR® Low Binding Plates, THERMO SCIENTIFIC™ NUNC™ Edge 2.0 Plates, SARSTEDT Low Binding Cell Culture Plates, and FISHERBRAND™ Low Binding Plates.

Methods of generating organoids are known in the art. In an aspect, the methods may use an extracellular matrix to develop the organoids. Non-limiting examples of extracellular matrices for use in the disclosed method include solubilized basement membrane (e.g., MATRIGEL®), collagen I, laminin, fibronectin, and hyaluronic acid-based matrices. In an aspect, the disclosed method does not require an exogenous extracellular matrix. In an aspect, the disclosed fibroblasts and/or the thymus extract may serve as a source of extracellular matrix, thereby foregoing the need for additional anchor.

In an aspect, the disclosed organoid may be cultured in any suitable media. The medium can be a serum-containing or serum-free medium, or xeno-free medium. From the aspect of preventing contamination with heterogeneous animal-derived components, serum can be from the same animal, and in an aspect, the same subject. The serum-free medium refers to medium with no unprocessed or unpurified serum and accordingly, can include medium with purified blood-derived components or animal tissue-derived components (such as growth factors). The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thioglycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco). In certain aspects, the medium may comprise one, two, three, four, five, six, seven, eight, nine, ten, 11, 12, 13, 14, 15, 16, 17, 18, 19, 20 or more of the following: vitamins such as biotin; DL Alpha Tocopherol Acetate; DL Alpha-Tocopherol; vitamin A (acetate); proteins such as BSA (bovine serum albumin) or human albumin, fatty acid free Fraction V; catalase; human recombinant insulin; human transferrin; superoxide dismutase; other components such as corticosterone; D-galactose; ethanolamine hc1; glutathione (reduced); l-carnitine hc1; linoleic acid; linolenic acid; progesterone; putrescine 2hc1; sodium selenite; and/or t3 (triiodo-L-thyronine). In specific aspects, one or more of these may be explicitly excluded.

In some aspects, the medium further comprises one or more vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triiodo-L-thyronine, or combinations thereof. In some aspects, the medium comprises one or more of the following: a B-27® supplement, xeno-free B-27® supplement, GS21™ supplement, or combinations thereof. In some aspects, the medium comprises or further comprises amino acids, monosaccharides, and inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triiodo-L-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), antioxidant substances, 2-mercaptoethanol, pyruvic acid, buffering agents, and/or inorganic salts. In specific aspects, one or more of these may be explicitly excluded. In an aspect, the medium may also contain growth one or more nucleic acids, cytokines, chemokines, growth factors, transcription factors, epigenetic factors, hormones, or any combination thereof. In an aspect, the medium may further contain agents to activate immune cells, for example thymocytes.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, pg/ml, mg/ml, or any range derivable therein.

The culturing conditions can vary depending on the specific method and components of the culture. In some aspect, the organoid may be cultures at a fixed or variable temperature ranging from about 20° to about 40° C., or about 25° C. to about 38° C., or about 37° C. In an aspect, the organoid may be cultured at a fixed, or variable carbon dioxide concentration ranging from 1-10%, for example, about 1%, about 2%, about 3%, about 4%, about 5%, about 6%, about 7%, about 8%, about 9%, or about 10% carbon dioxide concentration. In an aspect, the oxygen tension can range from 1-20%, for example about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% and any combination of ranges derivable therein.

In some aspects, organoids may be cultured for at least between about 10 days and about 40 days, for at least between about 15 days and about 35 days, for at least between about 15 days and 21 days, such as for at least about 15, 16, 17, 18, 19 or 21 days. In some aspects, the organoid of the disclosure may be cultured for no longer than 60 days, or no longer than 50 days, or no longer than 45 days. The organoid may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days.

In specific aspects, the cells or organoids of the disclosure are specifically formulated. They may or may not be formulated as a cell suspension. In specific cases they are formulated in a single dose form. They may be formulated for systemic or local administration. In some cases, the cells are formulated for storage prior to use, and the cell formulation may comprise one or more cryopreservation agents, such as DMSO (for example, in 5% DMSO), methylcellulose, ethylene glycol, or any combination thereof. In some aspects, the organoid may be cryopreserved for at least a day, at least a week, at least 2 weeks, at least 1 months, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years or more before use.

### Generation of Engineered Cells

The one or more cells, including fibroblasts, TEC and/or thymocytes may be engineered as disclosed herein above. Genetic modification methods or compositions may be used to introduce nucleic acids into cells, edit genomic DNA or transcripts using gene editing, homologous and non-homologous recombination, TALENS, CRISPR, zinc finger nucleases or any combination thereof. These genetic modifications may introduce exogenous genes, delete, or mutate endogenous genes, introduce selection or identification markers or any combination thereof. These methods are known in the art and may be used in isolation or a combination of these methods may be used to obtain cells with desired properties.

### Method of Using

The current disclosure also encompasses methods of using the cell mixes or the organoids disclosed herein. In an aspect, the method may comprise a method of treating a disease or disorder in a subject in need thereof, the method comprising administrating to the subject the disclosed cells, the cell pre-mix or the organoid. Also, provided herein is a method of regenerating thymus tissue in a subject, comprising administrating to the subject the disclosed cells, the cell pre-mix or the organoid.

In an aspect, effective amounts of disclosed cells, the cell pre-mix or the organoid as prepared in methods encompassed by the disclosure are administered to an individual for a therapy or prevention of one or more medical conditions. In specific aspects, the cells are administered to a subject diagnosed with or suspected of having an immune deficiency, an athymic disease (DiGeorge syndrome, congenital athymia, severe combined immune deficiency, FOXN1 deficiency, CHARGE Syndrome, diabetic embryopathy, thymoma and thymic cancer associated diseases), acute or chronic thymic atrophy, thymic involution, cancer, autoimmune disease, infections or any combination thereof.

In an aspect, the disclosure provide methods for co-administration of the one or more fibroblasts, with one or more additional cells disclosed herein, such that they stimulate thymus function. In a specific aspect, methods are provided for co-administration of disclosed compositions with one or combination of growth factors, chemokines, and/or cytokines. The disclosed compositions may be treated under conditions to reduce immunogenicity, to stimulate vascular endothelial growth factor (VEGF) production by the introduced compositions or from endogenous cells under regulatory control of the individual thereby leading to tissue regeneration and revascularization of the thymus.

The treatment provided herein may comprise administration of a therapeutic compositions (e.g., organoid, premixed fibroblasts and dissociated thymus cells, premixes comprising fibroblasts, TEC, TEPs, and/or thymocytes, derivatives of cell, exosomes from fibroblasts, agents, etc., mixed with organoids or cells, alone or in combination). Treatment may be administered in any suitable manner known in the art.

In an aspect, the treatment may be provided in combination with one or more additional therapies. For example, a first and second treatment may be administered sequentially (at different times) or concurrently (at the same time). In some aspects, the first and second treatments are administered in a separate composition. In some aspects, the first and second treatments are in the same composition. The different therapies may be administered in one composition or in more than one composition, such as 2 compositions, 3 compositions, or 4 compositions. Various combinations of the compositions may be employed. The therapeutic compositions (e.g., organoid, premixed fibroblasts and dissociated thymus cells, premixes comprising fibroblasts, TEC, TEPs, and/or thymocytes, derivatives of cell, exosomes from fibroblasts, agents, etc., mixed with organoids or cells, alone or in combination) of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by surgery, by inhalation, intrathecally, intraventricularly, or intranasally. In an aspect, the therapy is administered via intravenous, intraarterial, subcutaneous, intraperitoneal, intramuscular, intrathymic or perithymic route. In some aspects, the therapy is administered intravenously. In some aspects, the composition is administered directly to the thymus. In some aspects, the therapy is not administered intravenously, intramuscularly, subcutaneously, topically, orally, transdermally, intraperitoneally, intraorbitally, by implantation, by surgery, by inhalation, intrathecally, intraventricularly, or intranasally.

In an aspect, the thymic organoid can be transplanted or injected into a living mammal within about 10 hours to about 72 hours of organoid culture establishment. In an aspect, the thymic organoid can be transplanted and or injected at about 10 hours to about 15 hours, 15 hours to about 20 hours, 20 hours to about 25 hours, 25 hours to about 30 hours, 30 hours to about 45 hours, 45 hours to about 50 hours, 55 hours to about 60 hours, 60 hours to about 65 hours, 65 hours to about 70 hours, 70 hours to about 75 hours after establishment of the organoid. In an aspect, the organoid can be cryopreserved in commonly used cell cryopreservation medium and used at a future date. In an aspect, the implantation or injection of the organoid gives rise to viable and functional T cells. In an aspect, the implanted thymic organoid may express one or more exogenous human major histocompatibility complex, exogenous co-stimulatory molecules, exogenous biological molecules, or genes that alter T cell differentiation, function, or activation.

In some aspects, the therapy is combined with one or more additional therapies, for example chemotherapy, androgen deprivation therapy, immune activation therapy, checkpoint blockade therapy, or pain therapy. In an aspect, the additional therapies may be administered concurrently or staggered. In an aspect, the one or more additional therapies may be administered orally, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of pg/kg or mg/kg of body weight can be converted and expressed in comparable concentration units of pg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some aspects, between about 10⁵ and about 10¹³ cells per 100 kg are administered to a subject per infusion. In some aspects, between about 1.5×10⁶ and about 1.5×10¹² cells are infused per 100 kg. In some aspects, between about 1×10⁹ and about 5×10¹¹ cells are infused per 100 kg. In some aspects, between about 4×10⁹ and about 2×10¹¹ cells are infused per 100 kg. In some aspects, between about 5×10⁸ cells and about 1×10¹¹ cells are infused per 100 kg. In some aspects, a single administration of cells/organoid is provided. In some aspects, multiple administrations are provided. In some aspects, multiple administrations are provided over a period of time. In some aspects, 3-7 administrations are provided over the course of 3-7 consecutive days. In some aspects, 5 administrations are provided over the course of 5 consecutive days. In some aspects, a single administration of between about 10⁵ and about 10¹³ cells per 100 kg is provided. In some aspects, a single administration of between about 1.5×10⁸ and about 1.5×10¹² cells per 100 kg is provided. In some aspects, a single administration of between about I×10⁹ and about 5×10¹¹ cells per 100 kg is provided. In some aspects, a single administration of about 5×10¹⁰ cells per 100 kg is provided. In some aspects, a single administration of 1×10¹⁰ cells per 100 kg is provided. In some aspects, multiple administrations of between about 10⁵ and about 10¹³ cells per 100 kg are provided

In some aspects, the thymus organoid may be used to differentiate and/or expand T cells reacting to a single or multiple antigens, in vitro and/or in vivo. These T cells may be isolated and administered to a subject without any polarization. In some aspects, the T cells are generated in vivo by the thymus organoid. In some embodiments, the T cells may be exposed to cytokines that are known to polarize towards cytotoxicity, Th1, Th2, Th17, Treg, Th9 and any combinations thereof. In an aspect, the thymic organoid may be used to generate T cells with a diverse TCR repertoire. These polarization approaches have been described in the art. In an aspect, the organoid may expresses Keratin 5, Keratin 8, FOXN1, beta 5t, DLL4, IL-7, SCF, IL-2 In an aspect, the thymic organoid does not require the addition of cytokines such as IL-7, FLT3L, SCF, IL-2. In an aspect, the thymic organoid can be used to generate thymic emigrant cells, immature T cells, mature T cells, antigen-specific T cells, chimeric antigen receptor (CAR)-T cells, CAR cells, regulatory T cells, cytotoxic T cells, invariant T cells, intraepithelial lymphocytes, gamma-delta T cells.

In some aspects, a thymocyte is engineered to express a CAR and/or TCR targeting an antigen associated with a disease or disorder. The antigen can be a tumor associated antigen. The antigen can be gp100, MAGE-A, MART-1, HER2/neu, hTERT, CEA, p53, CD19, CD20, GPC3, NYESO-1, MUC-1, CA-125, CA19-9, GD2, HPV (L1/E6,E7), HBV, SV40, CDk-4, beta-catenin, Mesothelin, PSMA, PTD52, Tyrosinase, PSA, PAP, EBV. In some aspects, a thymocyte expresses a TCR targeting gp 100 (PMEL), MAGE-A, MART-1, HER2/neu, hTERT, CEA, p53, CD19, CD20, GPC3, NYESO-1, MUC-1, CA-125, CA19-9, GD2, HPV (L1/E6,E7), HBV, SV40, CDk-4, beta-catenin, Mesothelin, PSMA, PTD52, Tyrosinase, PSA, PAP, EBV.

In an aspect, The thymic organoid can be supplied with all instrumentation required for the procedure in a kit form.

### EXAMPLES

### Example 1: Generation of Thymus-Organoids Using Dissociated Thymus

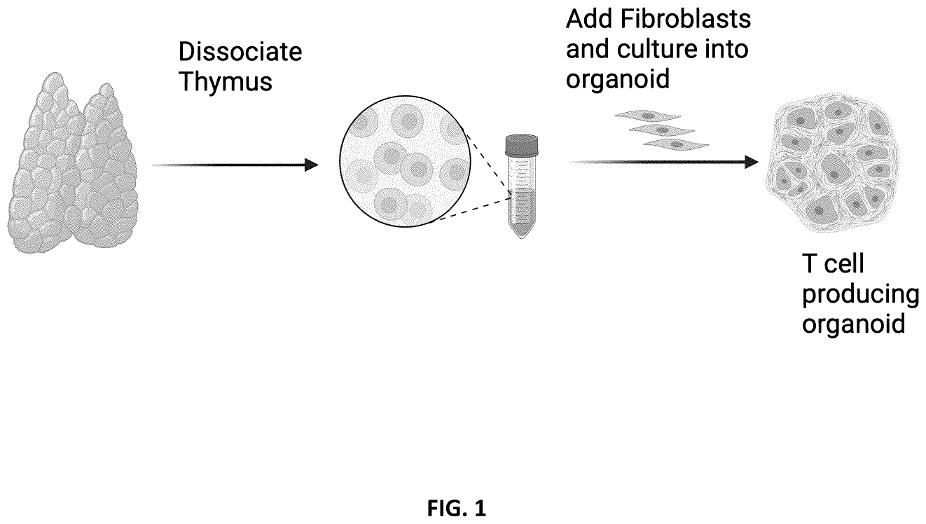

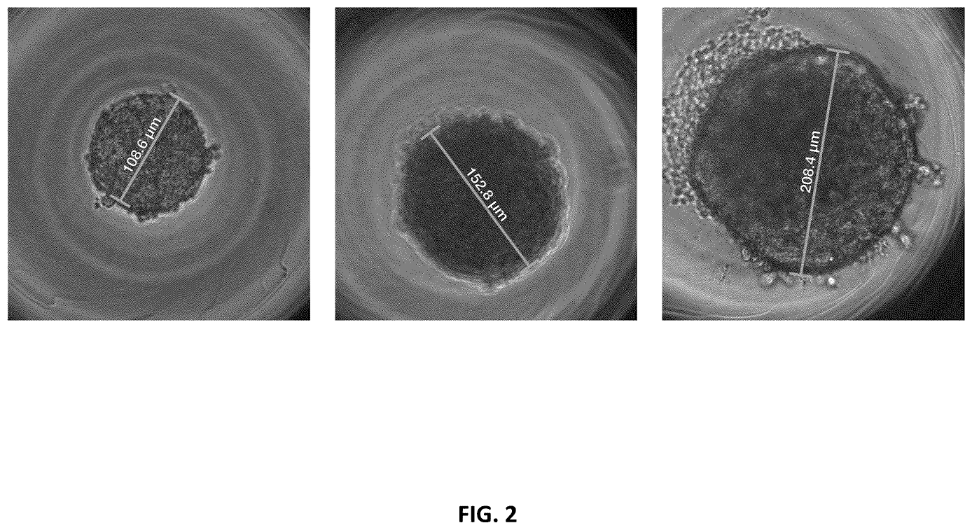

In an aspect, the current disclosure provides a method of generating thymus organoids. FIG. 1 provides a schematic of the general method used to develop the organoids. Briefly, isolated and/or cultured fibroblasts obtained by any method known in the art may be mixed with isolated or cultured thymic cells, and allowed to grow in defined culture conditions on a low adhesion surface to obtain a T-cell producing organoid. For example, 1×10⁶ fibroblasts can be combined with combined with 3×10⁶ thymocytes and cultured in a 24 well microcavity plate (e.g., ELPLASIA® plate). Using this method organoids could be generated in a size range of up to 2,000 μm. FIG. 2 provides some examples of thymus organoids developed using this method varying in size from about 100-200 μm.

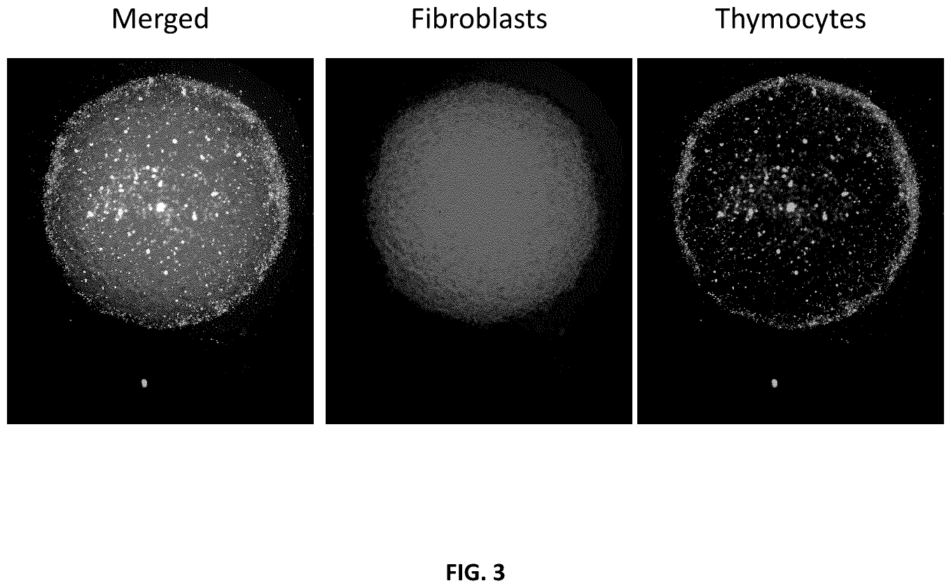

Fibroblasts were labelled with a viability dye (e.g., Calcein Am dye), Thymocytes were labelled with a cell proliferation dye (e.g., EFLOUR™ 670 dye), then the two populations were combined at a ratio of 1:3 respectively. After about 24 hours (hrs.) of culture, the organoids were imaged. FIG. 3 demonstrates the integration of the two cell types into a thymus-mass of cells (e.g., an organoid).

### Example 2: Validation of the Formation of Functional Thymus Thymic Organoids

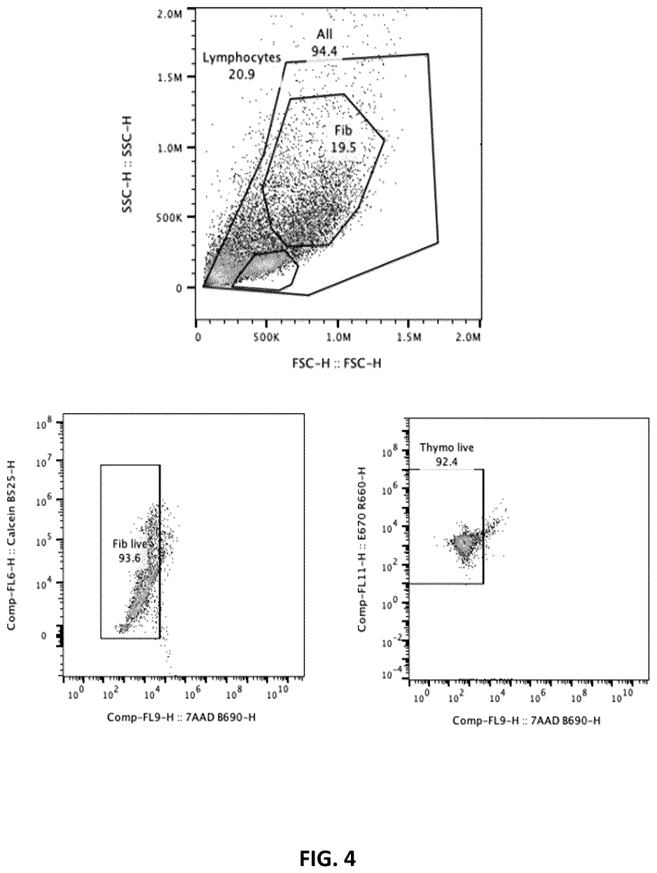

Organoids were cultured for 3 weeks and subjected to flow cytometric analysis to validate the disclosed method. Cells were initially gated on size and granularity (see FIG. 4, left panel). Clusters corresponding to lymphocytes and fibroblasts could be clearly seen. About 21% of the cells in the organoid corresponded to lymphocytes and about 20% to fibroblasts. Fibroblasts and thymocytes were further tested for viability using 7-Aminoactinomycin D (7-AAD) vital dye. 7-AAD is a membrane impermeant dye that is generally excluded from viable cells. Results show that about 93.6% of fibroblasts and 92.4% of thymocytes were viable.

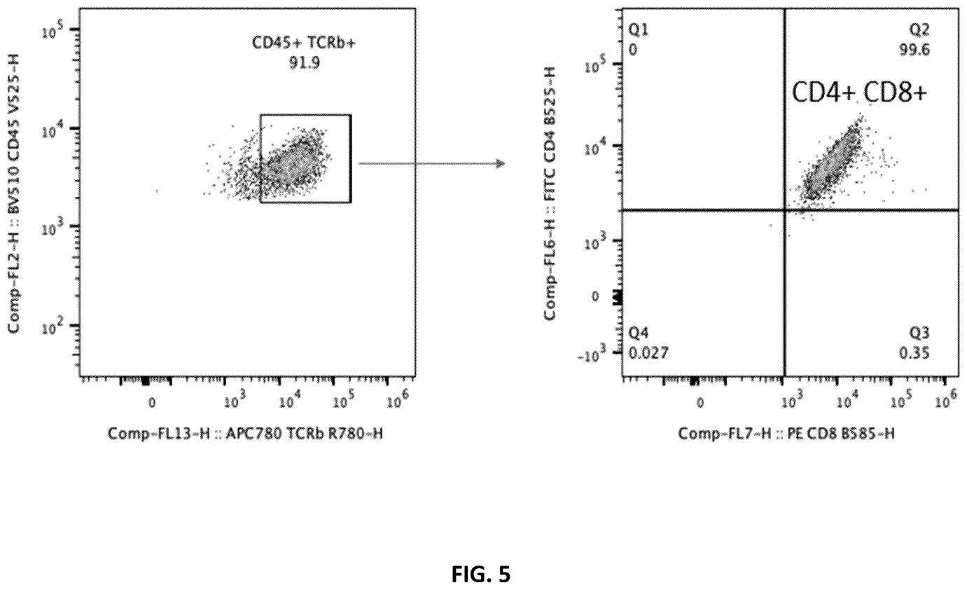

Lymphocytes were further gated for CD45 and TCR-b population demonstrating the T cell precursors had begun to rearrange the TCR, a critical step in T cell development. Furthermore, they have progressed to express CD8 and CD4 antigens on the cell surface, a stage termed as the double positive stage. As seen in FIG. 5, about 99.6% of the lymphocytes were double positive for CD4 and CD8, indicating undifferentiated T-cell lymphocytes (thymocytes) as expected from thymic organoids.

### Example 3: The Thymus-Organoids Were Viable After a Freeze-Thaw Cycle

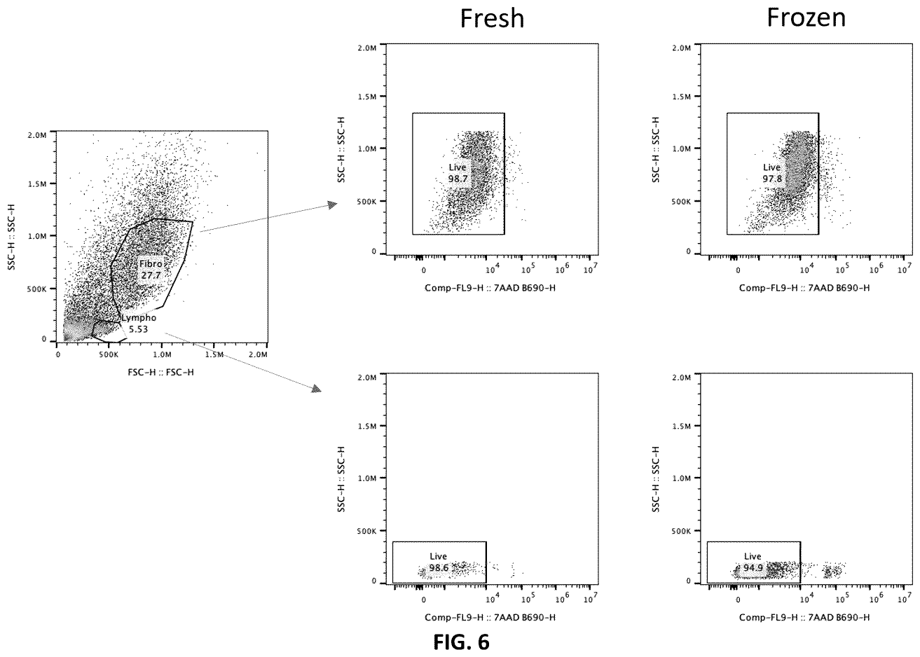

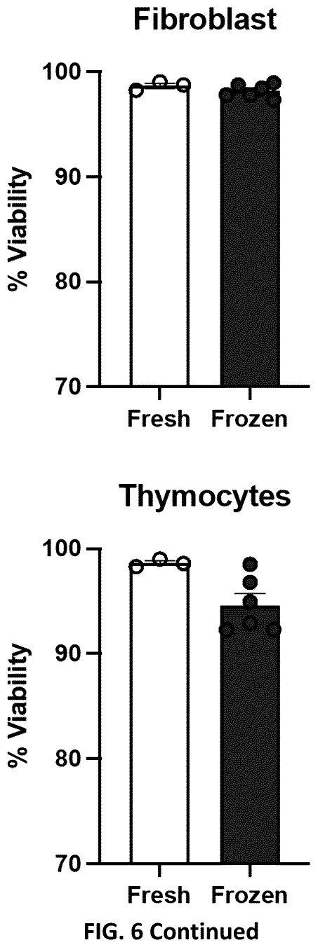

Next, to test if organoids could be stored prior to use, the organoid were cryopreserved at −80° C. using a cryopreservative mixture comprising of DMEM-F 12, 10% FBS, 1% Pen-strep and 10% DMSO or a commercial cryopreservation media (e.g., CRYOSTOR 10®). This is a commonly used formulation for cryopreservation of cells. The organoids were then dissociated into single cells using a cell detachment solution (e.g., ACCUMAX™) and tested for viability using 7-AAD as provided in Example 2. FIG. 6 shows that more than 95% of both the thymocytes and fibroblasts were viable after freezing.

### Example 4: In Vivo Injection of Thymus-Organoids in SCID Mice

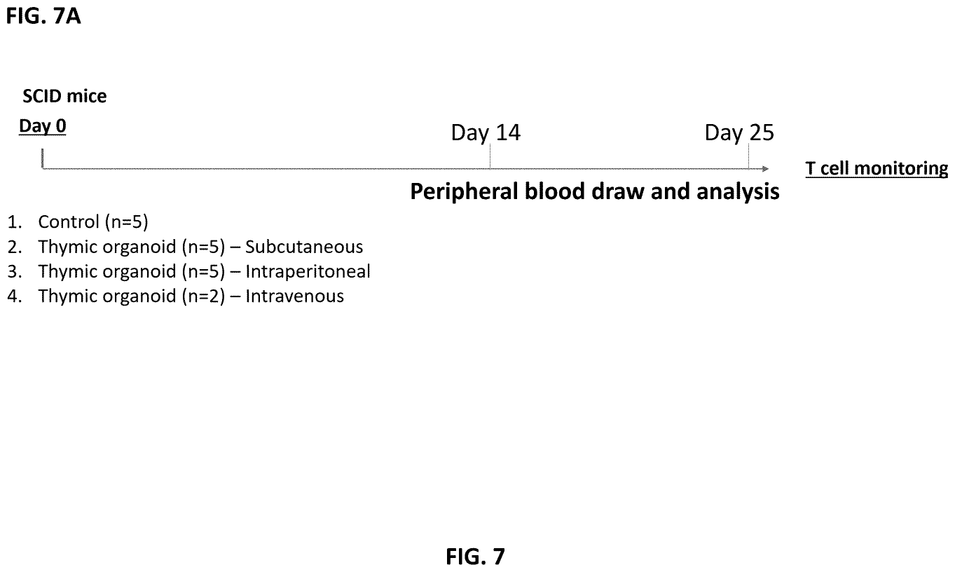

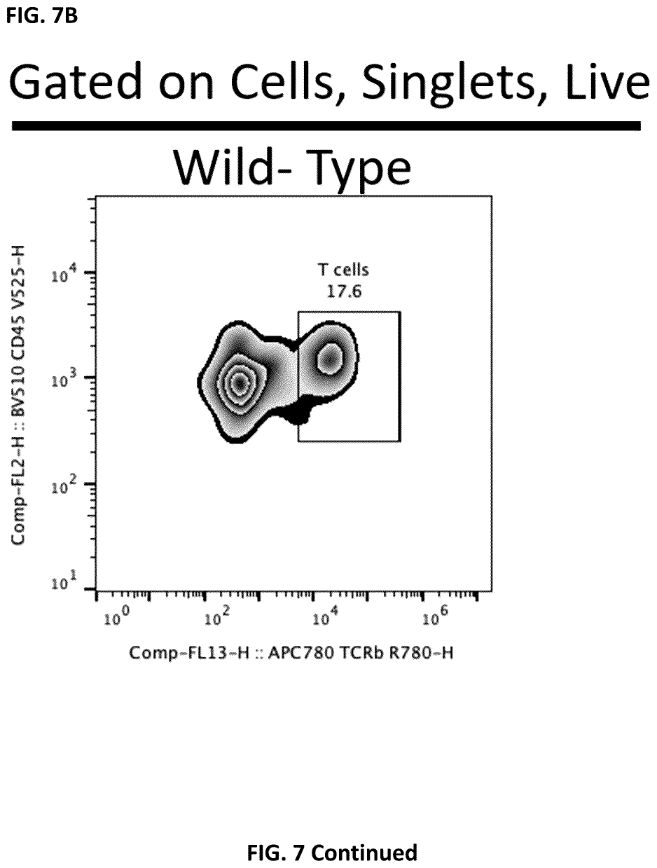

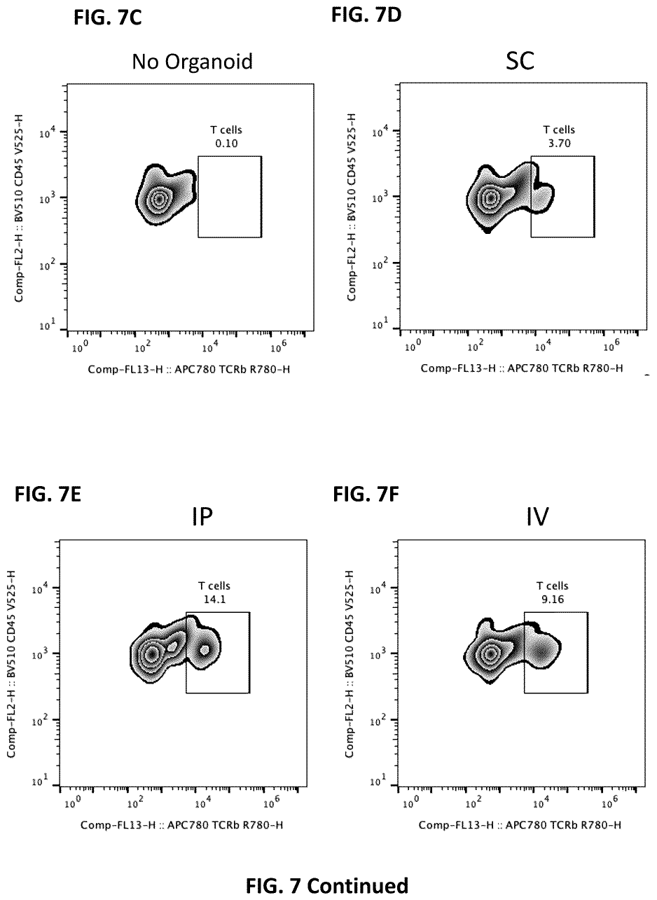

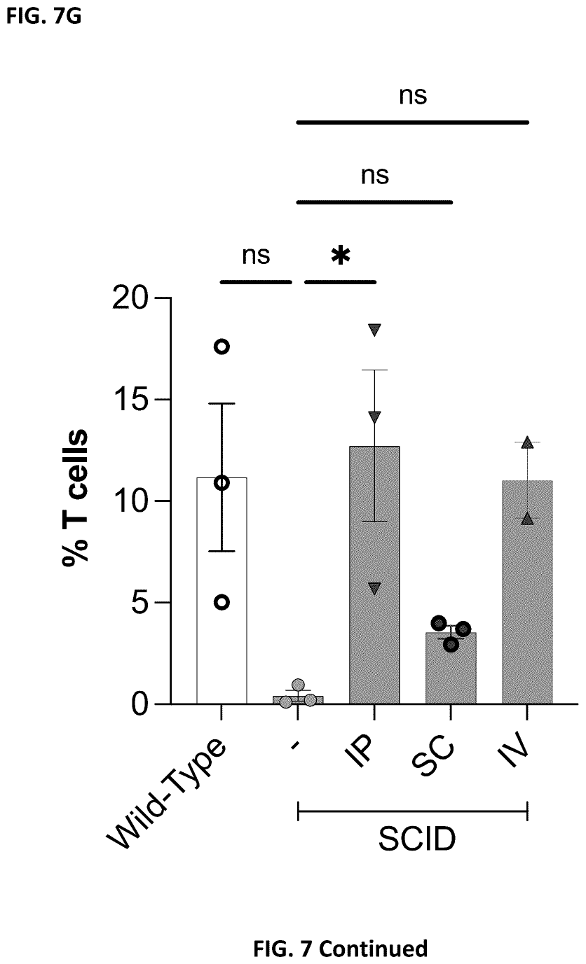

In vivo viability and efficacy of thymus-organoids was tested in SCID mice (CBySmn.Cg-Prkdcscid/J: JAX:001803). Five SCID mice were used for each set of experiments and injected using either subcutaneous (SC), intraperitoneal (IP), or intravenous (IV) routes. A set of 5 mice were used as control (non-injected). Peripheral blood was drawn and cells were subjected to flow cytometric analysis on day 14 and day 25. A schematic of the timeline is provided in FIG. 7A. Samples were tested for the presence of T-lymphocytes in each of the 5 groups of mice. TCR-β and CD 45, T-cell lymphocyte markers, were used in the flow cytometry experiments. As shown in FIG. 7B, wild-type mice have about 17.6% T-lymphocytes. On day 14, control mice showed less than 0.1% T lymphocyte, while all the three injected experimental groups showed higher than control T-cell population (see FIGS. 7C-7F; Ctrl: 0.1%, SC: 3.7%, IP: 14.1% and IV: 9.2%). FIG. 7G provides quantitation of the data.

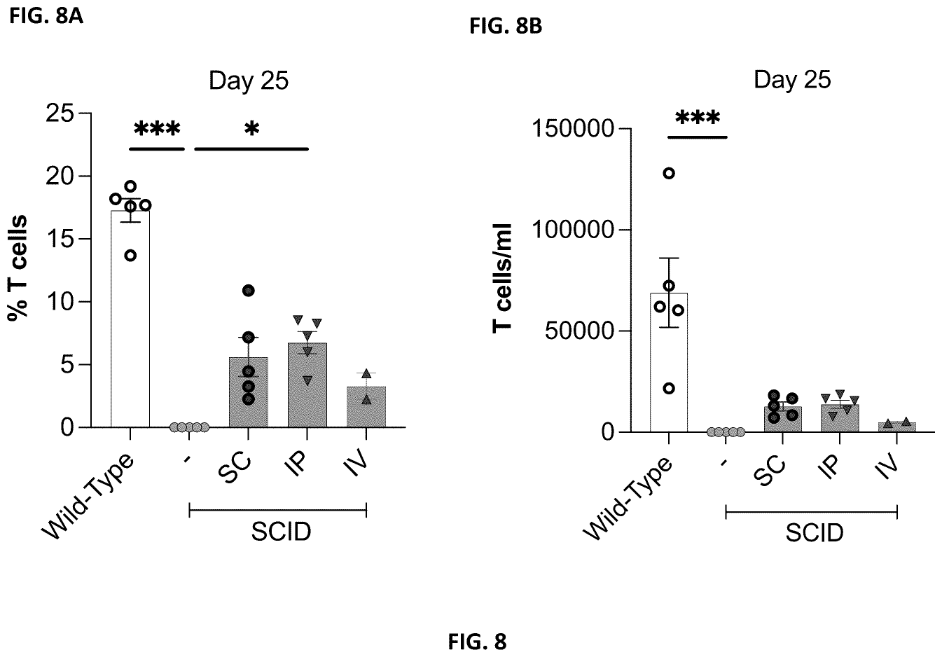

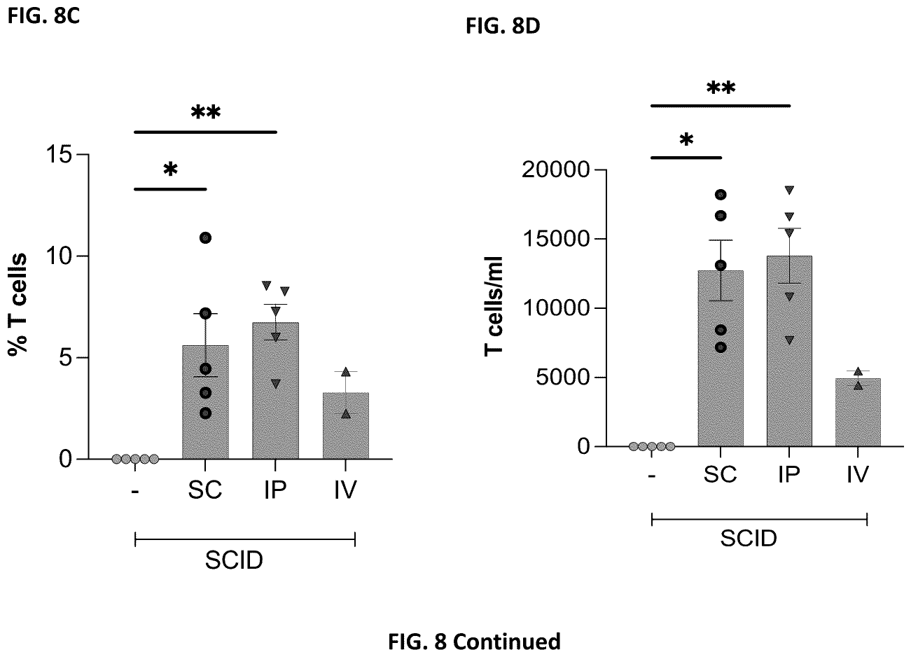

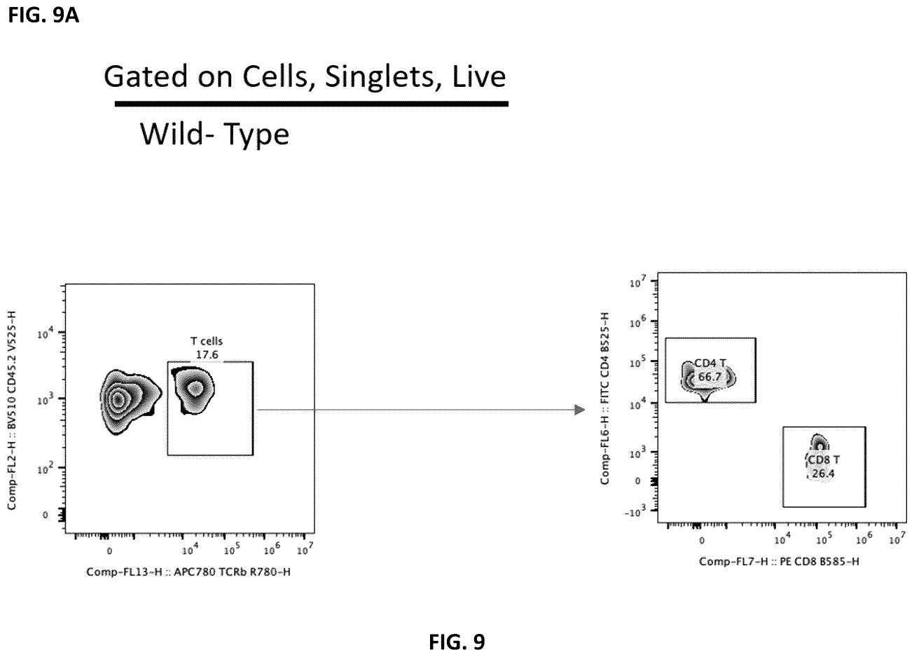

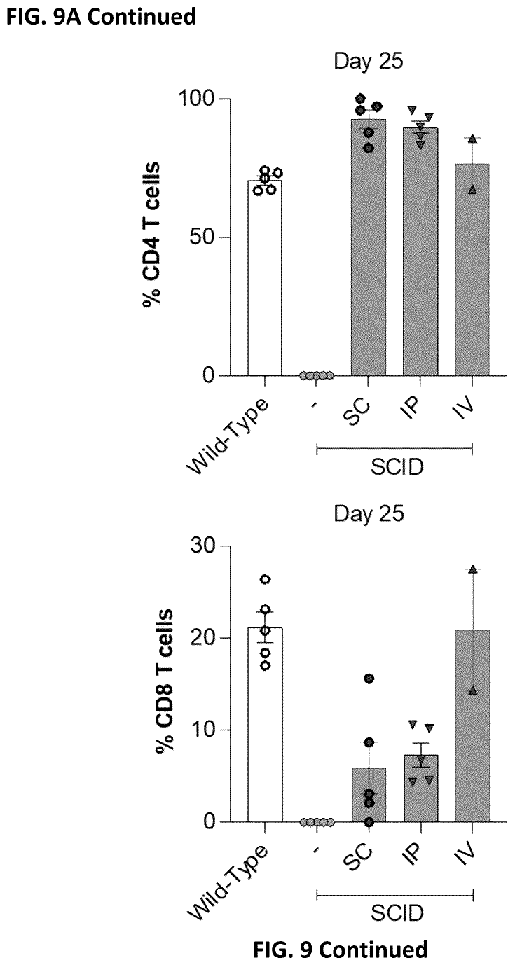

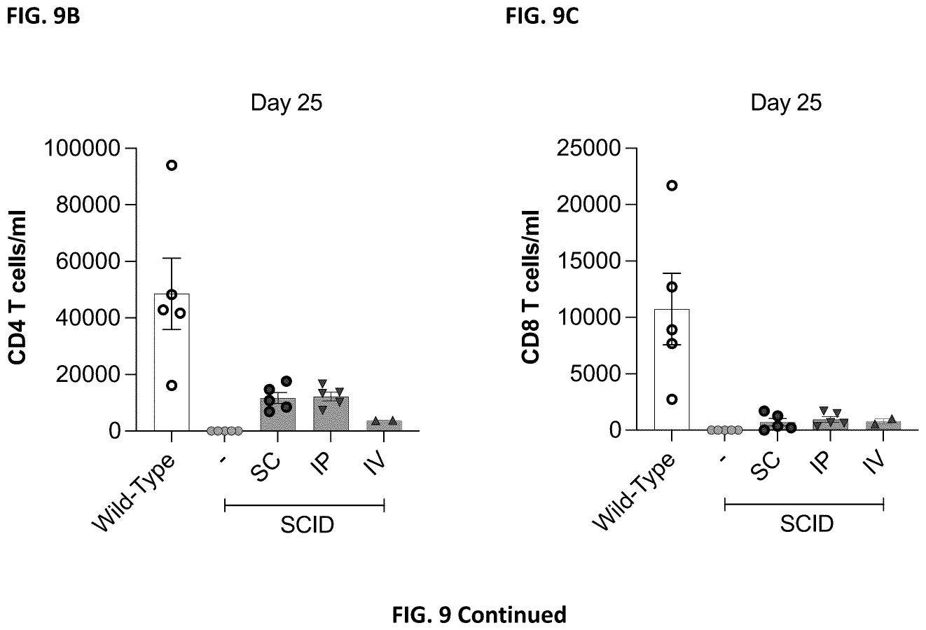

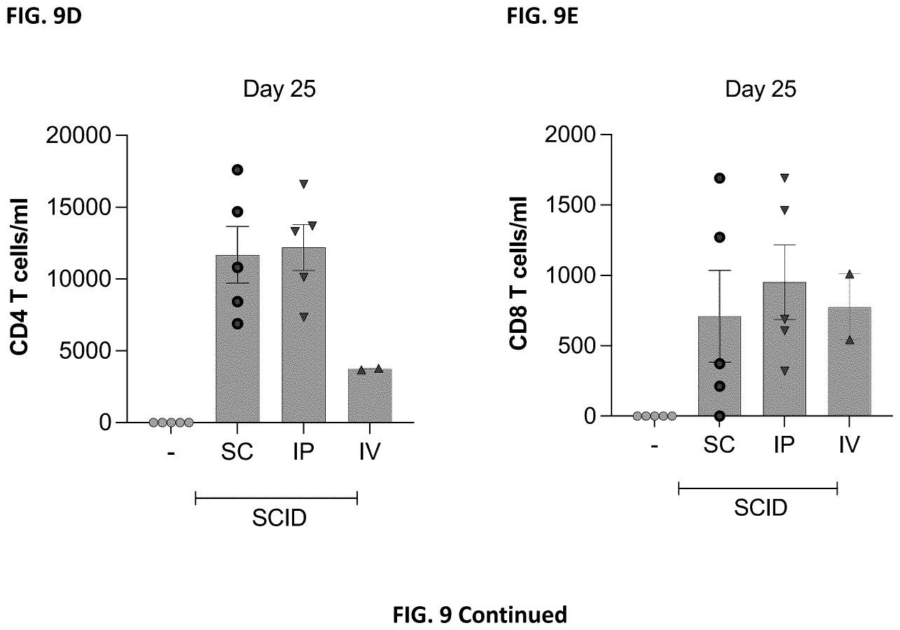

Peripheral blood drawn on day 25 after injection was similarly analyzed. FIG. 8A and FIG. 8B provides a quantitation of the data (% T-cells and T-cell count) in comparison to wild-type mice. FIG. 8C and FIG. 8D provides the same data without the wild-type data for clarity. Live T-cells from day 25 were further gated by CD4+ and CD8+ markers. Two distinct population of cells could be seen, corresponding to a differentiated populations of helper and cytotoxic T-cells (see FIG. 9A). Quantitation of the data is provided in FIGS. 9B-9C with wild-type and without wild-type in FIG. 9D-9E.

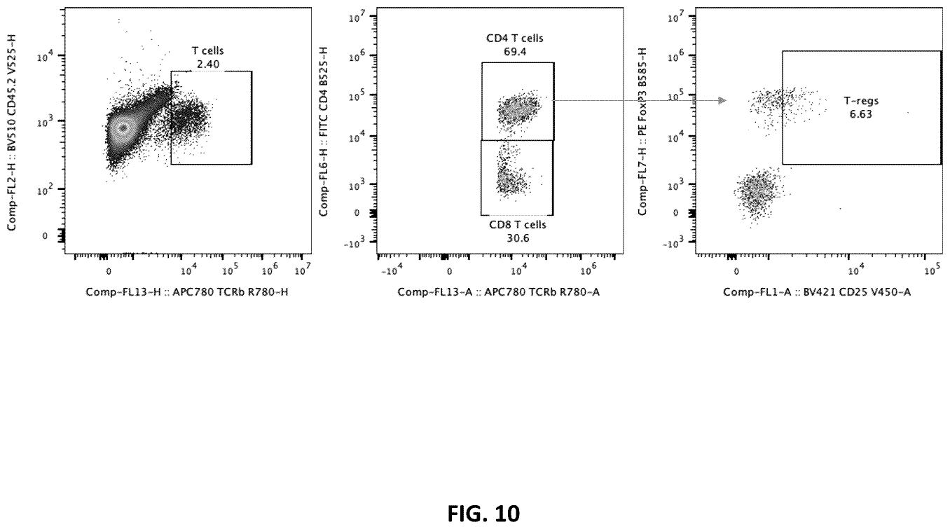

Peripheral blood was drawn on day 37 after injection and the presence of CD4+ CD25+ FOXP3+ regulatory T cells was analyzed. FIG. 10 shows the gating hierarchy which is gated on CD45+ TCRb+ (T cells), then gated on CD4+ T cells, then gated on CD25+ FOXP3+ cells which are commonly referred to as T-regs or regulatory T cells.

### Example 5: Generation of Spheroids From Engineered Fibroblasts Expressing FOXN1

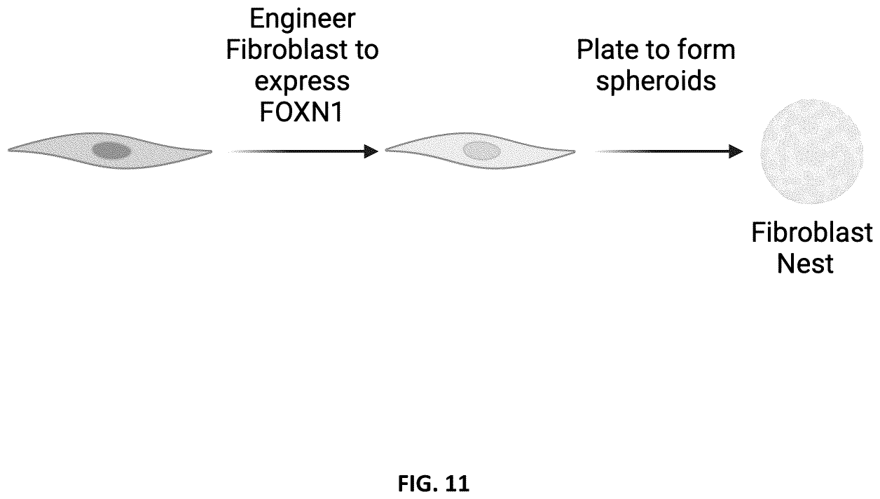

In an aspect, the disclosed method also encompasses the use of engineered fibroblasts into the thymus-organoids contemplated herein (see FIG. 11). Fibroblasts were genetically engineered to express FOXN1. Cells were grown in a normal cell culture environment (e.g., at 37° C., 5% carbon dioxide, 90% humidity) to 80% confluency for experiments. This method allows human dermal fibroblasts to obtain an 89-95% survival rate and have normal cell morphology, cell proliferation, and migration ability. These cells can then be integrated into organoids as provided in Example 1. Examples of the steps are as follows:
1. Isolate the cells, remove the medium, and wash twice with 1×PBS.
2. Dissociate cells with 0.25% trypsin for 3 minutes.
3. Suspend cells in a suitable medium (e.g., that comprises low glucose, human serum, non-essential amino acids, and/or L-glutamine, in specific aspects) to collect the cells.
4. Prepare microcavity flask (e.g., ELPLASIA®) by washing off air bubbles using 20% Isopropyl alcohol. Then wash twice with 1×PBS.
5. Adjust the cell concentration to 1×10⁶ cells/mL and resuspend to 24 ml of DMEM-F12 supplemented with FBS . Then gently add the cells to the microcavity flask (e.g., ELPLASIA®).
6. Place the culture plate into the normal cell culture environment for 72 hours.
7. Harvest the spheroids and spin down at 200 g for 7 minutes. Resuspend the spheroids to the desired concentration for immediate use or storage at −80 degrees.

### Example 6: Thymus Organoids Generate Functional T Cells

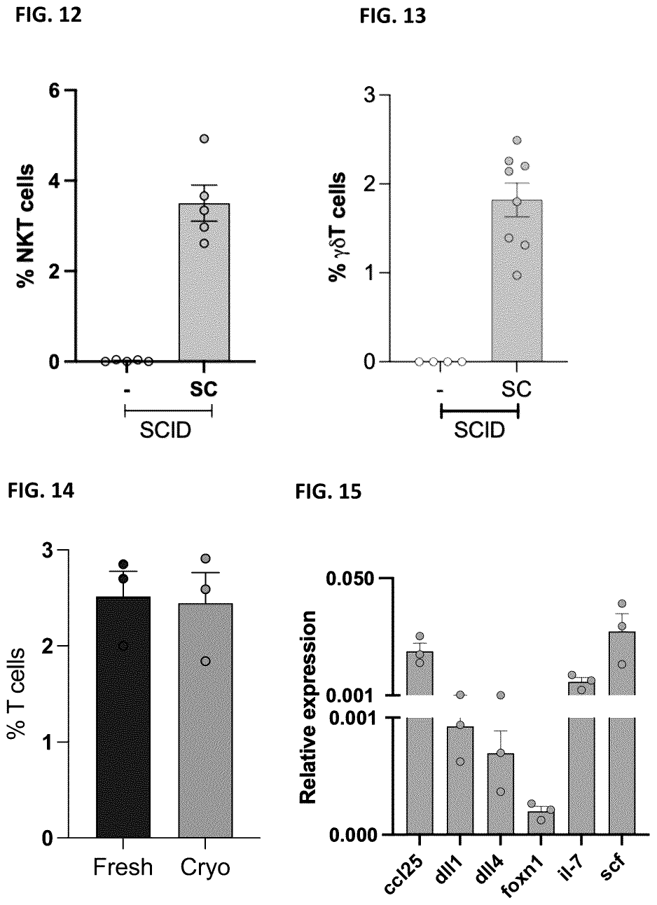

To assess the production of T cells (e.g., rare T cell subsets) by thymus organoids transplanted into SCID mice, the frequency of different T cell types was measured. Thymus organoids transplanted subcutaneously were capable of generating NKT cells at a frequency of about 3-4% compared (FIG. 12) and gamma-delta T cells at a frequency of about 1.5-2.5% (FIG. 13).

Thymus organoids were cryopreserved and injected into SCID mice to assess the effect of cryopreservation on the ability to generate T cell. Both fresh (i.e., non-cryopreserved) and cryopreserved thymus organoids produced comparable reconstitution of T cells (FIG. 14).

Reverse-transcription quantitative PCR was performed on thymus organoids. Gene expression was normalized to the house-keeping gene beta actin. The results showed that thymus organoids expressed key genes involved in T cell development and maturation (FIG. 15) indicating that thymus organoids can generate functional T cells.

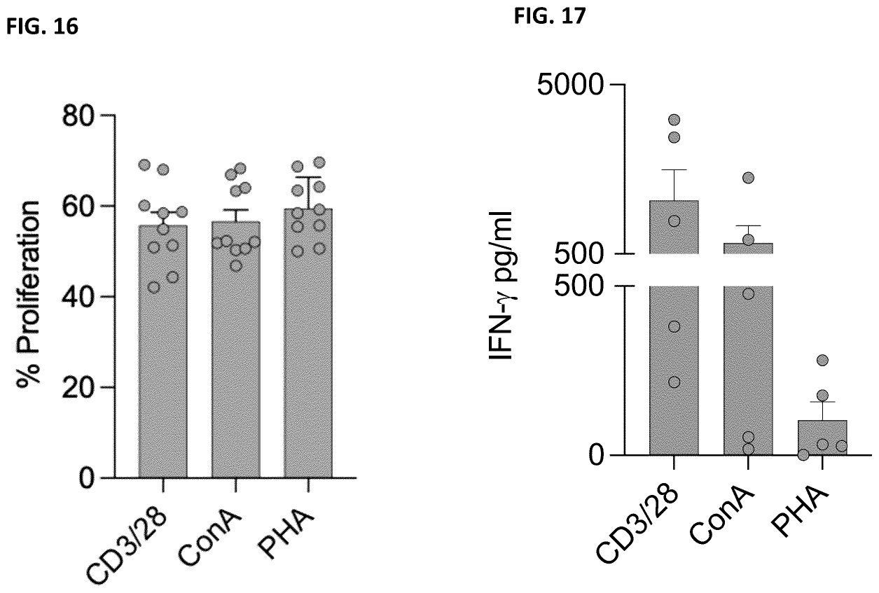

Thymus organoids were transplanted into SCID mice and spleens were isolated on day 28. Thymus organoids were stimulated with CD3/28, PHA, or Concanavalin A, and analyzed for T cell proliferation and expression of functional cytokines. The results showed that thymus organoids can produce functional T cells (FIG. 16), and T cells that express IFN-gamma in response to stimulation (FIG. 17).

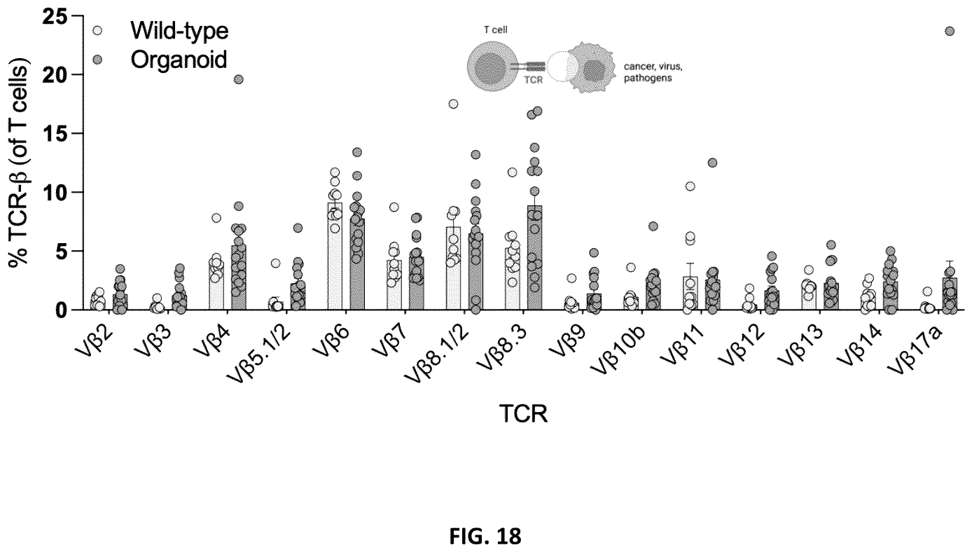

Thymus organoids were transplanted into SCID mice and spleens were isolated on day 28 for assessment of TCR-beta chain diversity. The results were compared to wild-type mice and showed that thymus organoids can generate T cells with diverse T cell receptor repertoire (FIG. 18).

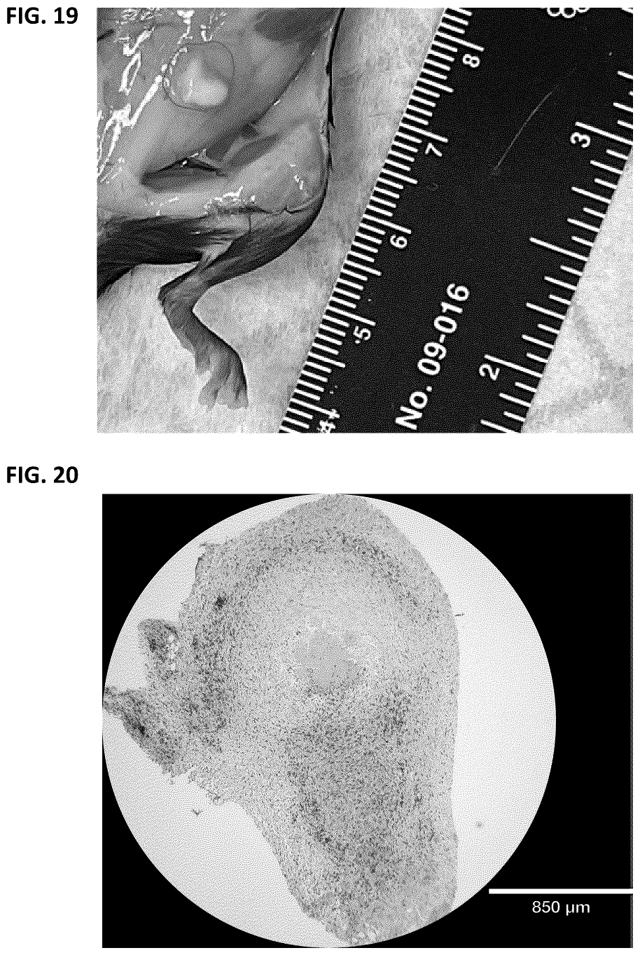

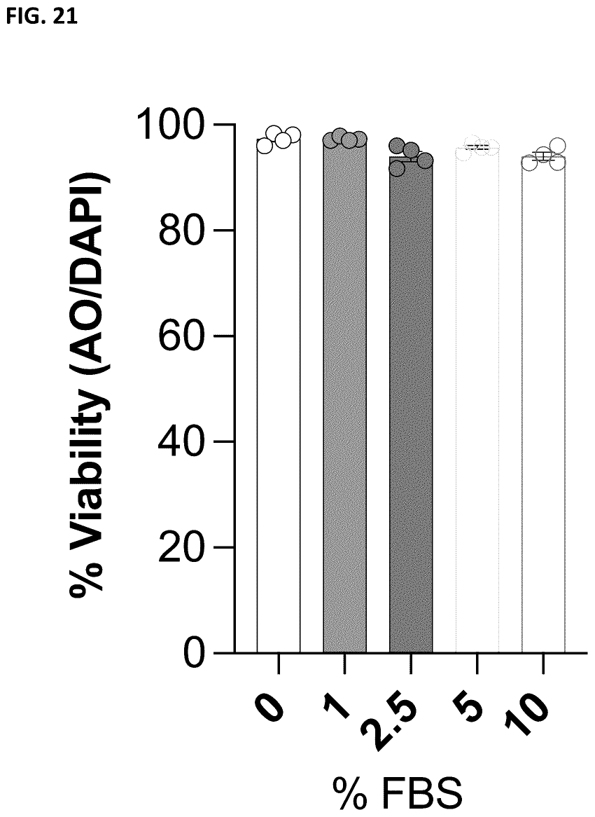

SCID mice were transplanted with 5000 thymus micro-organoids (˜150 μm diameter) and after 4 weeks developed into a single large thymus-like structure in vivo. These results showed that transplanted thymus organoids can form larger thymus-like structures in vivo (FIG. 19). FIG. 20 shows staining of CD3 in a thymus organoid that was transplanted into a SCID mouse. This demonstrated that T cell development and acquisition of the CD3 receptor occurred within the transplanted organoids.

Thymus organoids were grown in media containing different concentrations of serum (e.g., 0%, 1%, 2.5%, 5%, or 10% fetal bovine serum (FBS)). The results showed that thymus organoids can be grown in serum free condition and maintain high cell viability (FIG. 21).

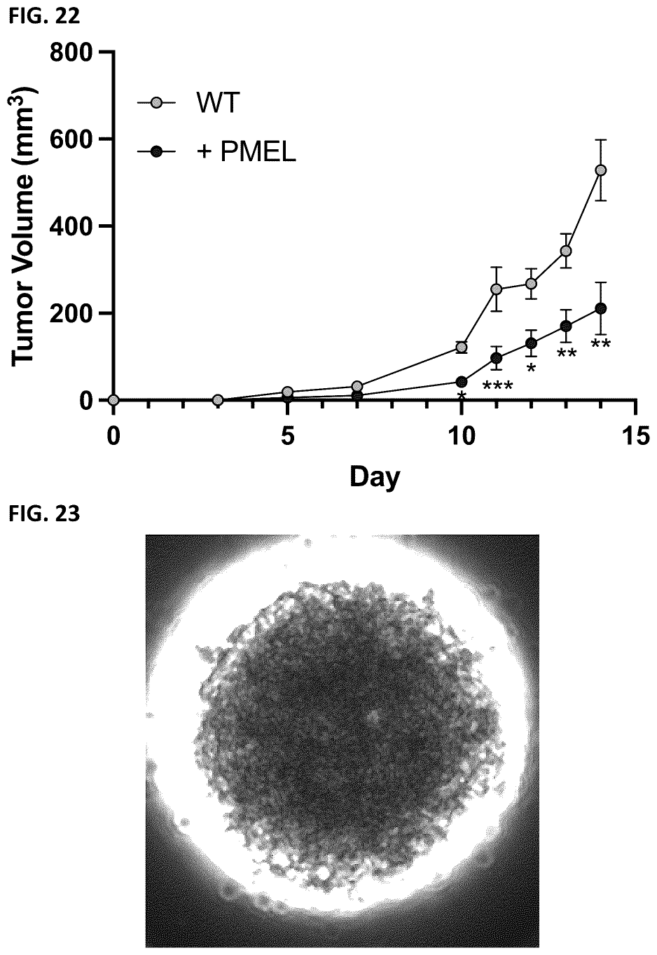

Thymus organoids were generated with thymocytes engineered to express T cell receptor that recognizes a gp100 antigen (PMEL), which is highly expressed in melanomas. These PMEL thymic organoids were transplanted into mice bearing B16 melanoma tumors. The results showed that thymus organoids can inhibit tumor growth (FIG. 22) indicating that antigen specific thymus organoids can produce T cells that can target antigens on tumors.

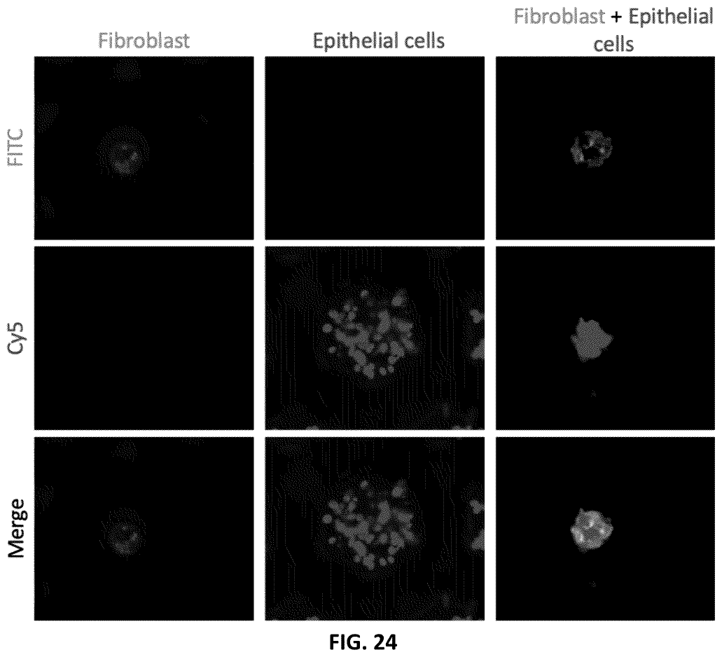

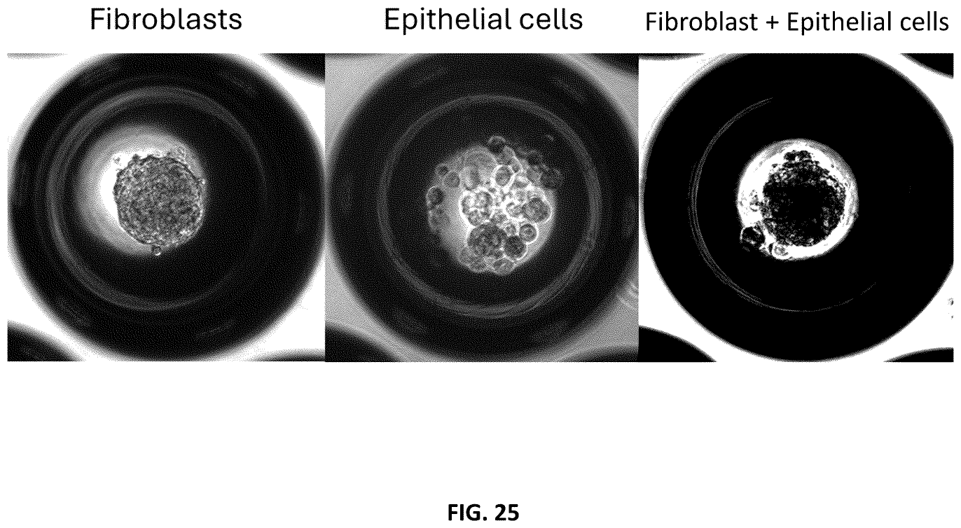

Epithelial cells (e.g., thymus epithelial cells) do not form spheroids on their own. Surprisingly, the inventors discovered that co-culture of fibroblasts and epithelial cells produced spheroids (FIG. 24; fibroblast labeled with CFSE/FITC, and epithelial cells labelled with E670 dye/Cy5; and FIG. 25 shows brightfield images). This data showed that fibroblasts contributed to the formation of the spheroids/organoids in culture.

1. A thymus organoid comprising one or more fibroblasts and/or one or more fibroblast-derived products; and
a) one or more thymic epithelial cells;
b) one or more thymocytes;
c) one or more T-cell progenitors; or
(d) a combination of (a), (b), and/or (c); and
wherein the ratio of the one or more fibroblasts to the one or more thymic epithelial cells, the one or more thymocytes, the one or more T-cell progenitors, or a combination thereof is in the range of about 0.0001:1 to 10000:1.

2. The thymus organoid of claim 1, wherein the one or more fibroblasts, one or more thymic epithelial cells, or the one or more thymocytes or any combination thereof are engineered cells.

3. The thymus organoid of claim 2, wherein the engineered fibroblasts, engineered thymic epithelial cells and/or the engineered thymocytes encode at least one exogenous gene product.

4. The thymus organoid of claim 3, wherein the at least one exogenous gene product comprise one or more of Notch ligands, one or more nucleic acids, one or more cytokines, one or more chemokines, one or more transcription factors, one or more epigenetic factors, one or more growth factors, one or more hormones, a fragment or derivative thereof, and a combination thereof.

5. The thymus organoid of claim 3, wherein the at least one exogenous gene product comprises DLL4, DLL1, DLL3, X-delta 2, JAG1, JAG2, FOXN1, IL-7, FLT-3L, SCF, CCL25, CXCL12, CXCL19, Lymphotoxin-alpha/beta, insulin-growth factor-1/2, fibroblast growth factor-7/10, FGF-21, HoxA1, Eya1, Pax9, Six1, Tbx1, Ripply3, E2F3, myc, miR-181a, Leptin receptor, ghrelin receptor, IL-22, a fragment thereof, a derivative thereof, or a combination thereof.

6. The thymus organoid of claim 2, wherein the engineered fibroblast, one or more thymic epithelial cells, and/or the one or more thymocytes, or any combination thereof, has a disrupted expression of one or more endogenous genes.

7. The thymus organoid of claim 1, wherein the thymic epithelial cells are thymic epithelial progenitor cells.

8. The thymus organoid of claim 1, wherein the fibroblasts, thymic epithelial cells, and/or thymocytes are differentiated from embryonic stem cells, induced pluripotent stem cells, hematopoietic stem cells, or progenitor cells.

9-12. (canceled)

13. The thymus organoid of claim 6, wherein the endogenous gene product is tissue factor encoded by an F3 gene, or wherein the one or more endogenous gene is an androgen receptor selected from Pgr, Nrli2, Ar, Esr1, Esr2, Gper1, Nr3c1, Nr3c2, or a combination thereof.

14. (canceled)

15. The thymus organoid of claim 6, wherein the one or more endogenous gene comprises an immune check point gene, or wherein the immune check point gene is selected from PD1, CTLA4, TIM3, TIGIT, CD96, BTLA, LAG3, or a combination thereof, and the engineered cell is a thymocyte.

16. (canceled)

17. The thymus organoid of claim 1, wherein the ratio of the one or more fibroblasts to the one or more thymic epithelial cells or the one or more thymocytes or a combination thereof is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1.

18-20. (canceled)

21. The thymus organoid of claim 1, wherein the thymocytes comprise one or more T-cell progenitor cells, one or more mature T-cells that are differentiated to target a single or multiple antigens, and/or one or more mature T cell.

22. (canceled)

23. The thymus organoid of claim 1, wherein the one or more fibroblasts, the one or more thymic epithelial cells, the one or more thymocytes, or any combination thereof, are activated cells.

24-28. (canceled)

29. The thymus organoid of claim 1, wherein the fibroblasts comprise fibroblasts from the skin, thymus, heart, blood vessels, bone marrow, skeletal muscle, liver, pancreas, brain, and/or foreskin.

30. A pharmaceutical composition comprising the thymus organoid of claim 1.

31. A method of generating a thymus organoid in vitro, the method comprising culturing one or more fibroblasts and/or one or more fibroblast-derived products; and
a) one or more thymic epithelial cells;
b) one or more thymocytes;
c) one or more T-cell progenitors; or
(d) a combination of (a), (b), and/or (c); and
in a culture medium, and on a low adhesion surface.

32-35. (canceled)

36. A method of regenerating thymus tissue in a subject in need thereof, the method comprising administering to the subject the thymus organoid of claim 1.

37. A method of treating a disease or disorder in a subject in need thereof, the method comprising administering to the subject the thymus organoid of claim 1.

38-40. (canceled)

41. A method of regenerating thymus tissue in a subject in need thereof, the method comprising administering to the subject one or more fibroblasts and/or one or more fibroblast-derived products; and
a) one or more thymic epithelial cells;
b) one or more thymocytes;
c) one or more T-cell progenitors; or
(d) a combination of (a), (b), and/or (c).

42-45. (canceled)

46. The thymus organoid of claim 1, wherein the thymus organoid does not comprise an exogenous extracellular matrix.
